(19) United States
(12) Patent Application Publication
(10) Pub. No.: US 2026/0183367 A1
(43) Pub. Date: Jul. 2, 2026

(54) TREATING PROTEINOPATHIES

(71) Applicant: Mayo Foundation for Medical Education and Research, Rochester, MN (US)

(72) Inventors: Wilfried O. Rossoll, Jacksonville Beach, FL (US); Bilal Khalil, Jacksonville, FL (US); Courtney Smith, Jacksonville, FL (US)

(21) Appl. No.: 19/128,283

(22) PCT Filed: Nov. 10, 2023
(86) PCT No.: PCT/US2023/079355

§ 371 Date: May 8, 2025

Related U.S. Application Data
(60) Provisional application No. 63/424,692, filed on Nov. 11, 2022.

(51) Int. Cl. A61K 38/17; A61P 25/28

(52) U.S. Cl. CPC A61K 38/1709; A61P 25/28

(57) ABSTRACT

This document relates to methods and materials for treating a mammal (e.g., a human) having, or at risk of developing, a proteinopathy. For example, one or more nucleoporin polypeptides (and/or nucleic acids designed to express a nucleoporin polypeptide) can be administered to a mammal (e.g., a human) having, or at risk of developing, a proteinopathy to treat the mammal.

### CROSS-REFERENCE TO RELATED APPLICATIONS

This application claims the benefit of U.S. Patent Application Ser. No. 63/424,692, filed on Nov. 11, 2022. The disclosure of the prior application is considered part of, and is incorporated by reference in, the disclosure of this application.

### SEQUENCE LISTING

This application contains a Sequence Listing that has been submitted electronically as an XML file named “07039-2174WOl.xml.” The XML file, created on Nov. 7, 2023, is 31,000 bytes in size. The material in the XML file is hereby incorporated by reference in its entirety.

### TECHNICAL FIELD

This document relates to methods and materials for treating a mammal (e.g., a human) having, or at risk of developing, a proteinopathy. For example, one or more nucleoporin polypeptides (and/or nucleic acids designed to express a nucleoporin polypeptide) can be administered to a mammal (e.g., a human) having, or at risk of developing, a proteinopathy to treat the mammal.

### BACKGROUND INFORMATION

Frontotemporal dementia (FTD) and amyotrophic lateral sclerosis (ALS) comprise a devastating continuum of progressive neurodegenerative diseases with the shared hallmark of TDP-43 pathology in 45% and 97% of cases, respectively. TDP-43 pathology is defined by the mis-localization of the predominantly nuclear RNA-binding protein TDP-43 into the cytoplasm, where it forms hyperphosphorylated and ubiquitinated detergent-insoluble aggregates. While mutations in the gene encoding TDP-43 can lead to ALS, the causes of TDP-43 proteinopathy in sporadic and other familial forms of ALS remain largely unknown.

### SUMMARY

This document provides methods and materials for treating a mammal (e.g., a human) having, or at risk of developing, a proteinopathy (e.g., a proteinopathy associated with aggregation of TAR DNA-binding protein 43 (TDP-43) polypeptides). Proteinopathies 5 associated with the aggregation of TDP-43 polypeptides can also be referred to as TDP-43 proteinopathies. As described herein, one or more nucleoporin polypeptides and/or fragments thereof (and/or nucleic acids designed to express a nucleoporin polypeptide and/or a fragment thereof) can be administered to a mammal (e.g., a human) having, or at risk of developing, a proteinopathy (e.g., a TDP-43 proteinopathy) to treat the mammal.

As described herein, nucleoporin polypeptides and fragments of nucleoporin polypeptides can reduce levels of insoluble TDP-43 (e.g., insoluble TDP-43 aggregates) present in proteinopathies. For example, nucleoporin 50 (Nup50) polypeptides and fragments of Nup50 polypeptides can prevent TDP-43 from undergoing pathological aggregation, can reverse formation of (e.g., can dissolve) insoluble TDP-43 aggregates, and can restore TDP-43 nuclear localization. Having the ability to reduce neurodegeneration in TDP-43 proteinopathies as described herein (e.g., by administering one or more nucleoporin polypeptides and/or fragments thereof and/or nucleic acids designed to express a nucleoporin polypeptide and/or a fragment thereof) provides a unique and unrealized opportunity to treat mammal having, or at risk of developing, a TDP-43 proteinopathy such as ALS.

In general, one aspect of this document features methods for treating a mammal having a TDP-43 proteinopathy. The methods can include, or consist essentially of, administering a Nup50 polypeptide or a fragment of the Nup50 polypeptide to a mammal having a TDP-43 proteinopathy. The Nup50 polypeptide or the fragment can be effective to reduce a symptom of the TDP-43 proteinopathy. The symptom can be deterioration in behavior and personality, depression, apathy, social withdrawal, mood swings, irritability, aggressiveness, changes in sleeping habits, wandering, loss of inhibitions, delusions, emotional blunting, compulsive or ritualistic behavior, changes in eating habits or diet, deficits in executive function, lack of insight, agitation, emotional instability, difficulty producing or comprehending spoken or written language, memory loss, and symptoms of motor neuron disease such as muscle weakness, muscle atrophy, fasciculations, spasticity, dysarthria, or dysphagia. The method can include identifying the mammal as being in need of the Nup50 polypeptide or the fragment prior to the administering step. The method can include administering the Nup50 polypeptide to the mammal. The method can include administering the fragment of the Nup50 polypeptide to the mammal. The fragment of the Nup50 polypeptide can consist of the amino acid sequence set forth in any one of SEQ ID NOs:3-11. The mammal can be a human. The TDP-43 proteinopathy can be frontotemporal dementia (FTD), amyotrophic lateral sclerosis (ALS), Alzheimer's disease, traumatic brain injury (TBI), chronic traumatic encephalopathy (CTE), limbic-predominant age-related TDP-43 encephalopathy (LATE), dementia with Lewy bodies (DLB), Parkinson's disease, Huntington's disease, argyrophilic grain disease (AGD), hippocampal sclerosis (HS), Guam ALS, Guam parkinsonism-dementia complex (G-PDC), Perry disease, facial onset sensory and motor neuronopathy (FOSMN), inclusion body myositis (IBM), oculopharyngeal muscular dystrophy (OPMD), or distal myopathies with rimmed vacuoles (DMRV). The administering can include intracerebral injection.

In another aspect, this document features methods for reducing aggregation of TDP-43 polypeptides in a central nervous system (CNS) of a mammal having a TDP-43 proteinopathy. The methods can include, or consist essentially of, administering an Nup50 polypeptide or a fragment of the Nup50 polypeptide to a mammal having a TDP-43 proteinopathy. The method can include administering the Nup50 polypeptide to the mammal.

The method can include administering the fragment of the Nup50 polypeptide to the mammal. The fragment of the Nup50 polypeptide can consist of the amino acid sequence set forth in any one of SEQ ID NOs:3-11. The method can include identifying the mammal as being in need of the Nup50 polypeptide or the fragment prior to the administering step. The mammal can be a human. The TDP-43 proteinopathy can be FTD, ALS, Alzheimer's disease, TBI, CTE, LATE, DLB, Parkinson's disease, Huntington's disease, AGD, HS, Guam ALS, G-PDC, Perry disease, FOSMN, IBM, OPMD, or DMRV. The administering can include intracerebral injection.

In another aspect, this document features methods for treating a mammal having a TDP-43 proteinopathy. The methods can include, or consist essentially of, administering nucleic acid encoding a Nup50 polypeptide or a fragment of the Nup50 polypeptide to the mammal, where the Nup50 polypeptide or the fragment is expressed by cells in a CNS of a mammal having a TDP-43 proteinopathy. The Nup50 polypeptide or the fragment can be effective to reduce a symptom of the TDP-43 proteinopathy. The symptom can be deterioration in behavior and personality, depression, apathy, social withdrawal, mood swings, irritability, aggressiveness, changes in sleeping habits, wandering, loss of inhibitions, delusions, emotional blunting, compulsive or ritualistic behavior, changes in eating habits or diet, deficits in executive function, lack of insight, agitation, emotional instability, difficulty producing or comprehending spoken or written language, memory loss, and symptoms of motor neuron disease such as muscle weakness, muscle atrophy, fasciculations, spasticity, dysarthria, or dysphagia. The method can include identifying the mammal as being in need of the nucleic acid prior to the administering step. The nucleic acid can encode the Nup50 polypeptide. The nucleic acid can encode the fragment of the Nup50 polypeptide. The Nup50 polypeptide can consist of the amino acid sequence set forth in any one of SEQ ID NOs:3-11 The nucleic acid can be in the form of a vector. The vector can be an adeno-associated virus (AAV) vector. The vector can be an expression plasmid. The mammal can be a human. The TDP-43 proteinopathy can be FTD, ALS, Alzheimer's disease, TBI, CTE, LATE, DLB, Parkinson's disease, Huntington's disease, AGD, HS, Guam ALS, G-PDC, Perry disease, FOSMN, IBM, OPMD, or DMRV. The administering can include intracerebral injection.

In another aspect, this document features methods for reducing aggregation of TDP-43 polypeptides in a CNS of a mammal. The methods can include, or consist essentially of, administering nucleic acid encoding a Nup50 polypeptide or a fragment of the Nup50 polypeptide to the mammal where the Nup50 polypeptide or the fragment is expressed by cells in the CNS of the mammal. The method can include identifying the mammal as being in need of the nucleic acid prior to the administering step. The nucleic acid can encode the Nup50 polypeptide. The nucleic acid can encode the fragment of the Nup50 polypeptide. The Nup50 polypeptide can consist of the amino acid sequence set forth in any one of SEQ ID NOs:3-11. The nucleic acid can be in the form of a vector. The vector can be an AAV vector.

The vector can be an expression plasmid. The mammal can be a human. The TDP-43 proteinopathy can be FTD, ALS, Alzheimer's disease, TBI, CTE, LATE, DLB, Parkinson's disease, Huntington's disease, AGD, HS, Guam ALS, G-PDC, Perry disease, FOSMN, IBM, OPMD, or DMRV The administering can include intracerebral injection.

In another aspect, this document features uses of a composition including a Nup50 polypeptide or a fragment of the Nup50 polypeptide to treat a mammal having a TDP-43 proteinopathy.

In another aspect, this document features a Nup50 polypeptide or a fragment of the Nup50 polypeptide for use in the preparation of a medicament to treat a mammal having a TDP-43 proteinopathy.

In another aspect, this document features a Nup50 polypeptide or a fragment of the Nup50 polypeptide for use in the treatment of a TDP-43 proteinopathy.

In another aspect, this document features uses of a composition including nucleic acid encoding a Nup50 polypeptide or a fragment of the Nup50 polypeptide to treat a mammal having a TDP-43 proteinopathy.

In another aspect, this document features nucleic acid encoding Nup50 polypeptide or a fragment of the Nup50 polypeptide for use in the preparation of a medicament to treat a mammal having a TDP-43 proteinopathy.

In another aspect, this document features nucleic acid encoding Nup50 polypeptide or a fragment of the Nup50 polypeptide for use in the treatment of a TDP-43 proteinopathy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Structure and function of an exemplary Nup50 polypeptide. FIG. 1A) A schematic of a Nup50 polypeptide include the following functional domains: an N-terminal importin-alpha/Nup153/chromatin interaction domain, a central importin-beta region with phenylalanine-glycine (FG) motifs that can interact with importin-alpha, and a C-terminal Ran binding domain. FIG. 1B) A schematic of a Nup50 polypeptide within the nuclear basket.

FIG. 2. A screen identified Nup50 polypeptides, Nup98 polypeptides, and Aladin polypeptides (arrowheads) as nucleoporins that reduced detergent-insoluble TDP-43-(mutant nuclear localization signal) mNLS polypeptide levels. GFP-tagged nucleoporins were coexpressed with red fluorescent protein mCherry-tagged TDP-43mNLS in HEK293 cells and RIPA-buffer-insoluble fractions were immunoblotted and stained for TDP-43 and β-actin (loading control). TDP-43 signal was normalized to loading control and compared to GFP control. n=3, p<0.05.

FIGS. 3A-3E. Nup50 polypeptides and Nup98 polypeptides reduced cytoplasmic TDP-43 aggregation. FIG. 3A) Schematics of TDP-43 constructs. FIGS. 3B and 3D). Fluorescence microscopy of mCherry-tagged Nup50 (FIG. 3B) and red fluorescent protein mScarlet-tagged Nup98 (FIG. 3D) coexpressed with GFP-tagged TDP-43 constructs in HEK293 cells. Hoechst DNA staining was used to trace nuclei. Scale bar=5 μm. FIG. 3C and FIG. 3E). Immunoblots stained for TDP-43 and β-actin (loading control) of RIPA-buffer-insoluble fractions of HEK293 cells overexpressing mCherry-tagged Nup50 (FIG. 3C top) and mScarlet-tagged Nup98 (FIG. 3E top). Quantification of TDP-43 signal normalized to loading control and compared to fluorescent control treatment (FIG. 3C bottom and FIG. 3E bottom). n=3, ****p<0.0001.

FIGS. 4A-4C. Nup50 polypeptides and Nup98 polypeptides reduced the accumulation of pathological hyperphosphorylated TDP-43. mCherry-tagged Nup50 was coexpressed with GFP-tagged TDP-43 constructs and immunocytochemistry was performed staining for TDP-43 phosphorylated at S409/410 (FIG. 4A). Scale bar=5 μm. Immunoblots of RIPA-buffer-insoluble fractions of HEK293 cells coexpressing GFP-tagged TDP-43 constructs with either mCherry-tagged Nup50 polypeptides (FIG. 4B) or mScarlet-tagged Nup98 polypeptides (FIG. 4C) stained for TDP-43 phosphorylated at S409/410 (pTDP^{409/410}) and β-actin (loading control). TDP-43 signal was normalized to loading control and compared to mCherry (FIG. 4B) or mScarlet (FIG. 4C). n=3, ***p=0.0001, ****p<0.0001.

FIGS. 5A-5C. The N-terminus of Nup50 was sufficient for reducing cytoplasmic TDP-43 aggregates. FIG. 5A) Schematic of Nup50 fragments and truncations (top) and AlphaFold protein structure prediction of Nup50 highlighting double alpha-helices within the smallest active fragment (bottom). FIG. 5B) Immunoblots of RIPA-insoluble fractions from HEK293 cells coexpressing mCherry-tagged Nup50 fragments and GFP-tagged TDP-mNLS were stained for TDP-43, mCherry, and β-actin (loading control). Arrowheads indicate constructs that decreased insoluble TDP-mNLS. FIG. 5C) Fluorescence microscopy showing representative examples for the coexpression of mCherry-tagged Nup50 constructs and GFP-tagged TDP-43 C-terminal fragment aa208-414 (TDP-CTF). Scale bar=5 μm.

FIGS. 6A-6C. NUP50 expression reduced TDP-43 aggregation. FIG. 6A) Schematic of GFP-tagged TDP-43mNLS, TDP-CTF, and short TDP splicing isoform of TDP-43 that lacks the C-terminal region (sTDP) constructs. FIG. 6B) Quantitative western blot analyses showed that NUP50 significantly reduced the RIPA-buffer insoluble levels of TDPCTF, TDP-43mNLS, and to a lesser degree sTDP, N=3; ****p<0.0001. FIG. 6C) mCherry-NUP50 suppressed pathological GFP-TDP-CTF and GFP-TDP-43mNLS aggregates and restored their nuclear localization, despite the lack of a functional NLS, while wild type GFP-TDP-43 remained nuclear and soluble.

FIGS. 7A-7C. The N-terminal fragment of Nup50 was sufficient to reduce TDP-43^{mNLS} aggregates. FIG. 7A) Top: Schematic of Nup50 domain structure and exemplary deletion constructs. Bottom: Predicted structure of Nup50 (AlphaFold). FIG. 7B) Quantitative western blot analyses showed that Nup50-N(aal-214) significantly reduced the RIPA-buffer insoluble polypeptide levels of TDP-43^{mNLS}. N=3; *p<0.05. FIG. 7C) mCherry-Nup50-N reduced pathological TDP-43^{mNLS} aggregated and restored its nuclear localization.

FIGS. 8A-8D. A comprehensive screen identified nucleoporins that can reduce insoluble TDP-43-mNLS protein levels. FIG. 8A) GFP-tagged nucleoporins were coexpressed with mCherry-tagged TDP-43-mNLS in HEK293 cells and RIPA-buffer-insoluble fractions were immunoblotted and stained for TDP-43 and β-actin (loading control). TDP-43 signal was normalized to loading control and compared to GFP control. A significant reduction of insoluble TDP-43-mNLS is observed for Nup50, Nup98, and Aladin. n=4. FIG. 8B) A schematic of an exemplary TDP-43 construct used with mutated nuclear localization sequence (TDP-43-mNLS). FIGS. 8C and 8D) representative images of western blots used to generate the quantified signal shown in FIG. 8A.

FIGS. 9A-9C. Nup50 reduces cytoplasmic TDP-43 aggregation. FIG. 9A) Schematic of TDP-43 constructs. FIG. 9B) Fluorescence microscopy showing coexpression of mCherry-tagged Nup50 with GFP-tagged TDP-43 constructs in HEK293 cells. Hoechst staining was used to trace nuclei, Scale bar=5 μm. FIG. 9C) Immunoblots of RIPA-buffer-insoluble fractions of HEK293 cells overexpressing mCherry-tagged Nup50 stained for TDP-43 and β-actin as a loading control. Quantification of TDP-43 signal normalized to loading control and compared to fluorescent control treatment. n=3, One-way ANOVA to test for significance, ****p<0.0001.

FIGS. 10A-10B. Nup50 reduces the accumulation of pathological hyperphosphorylated TDP-43. FIG. 10A) mCherry-tagged Nup50 was coexpressed with GFP-tagged TDP-43 constructs and immunocytochemistry was performed to stain TDP-43 phosphorylated at S409/410. Scale bar=5 μm. FIG. 10B) Immunoblots of RIPA-buffer-insoluble fractions of HEK293 cells coexpressing mCherry-tagged Nup50 with GFP-tagged TDP-43 constructs. Stained for TDP-43 phosphorylated at S409/410 and β-actin as a loading control. TDP-43 signal was normalized to loading control and compared to mCherry negative control. n=3, One-way ANOVA to test for significance ***p=0.0001, ****p<0.0001.

FIGS. 11A-11C. The N-terminus of Nup50 is required and sufficient for reducing cytoplasmic TDP-43 aggregates. FIG. 11A) Schematic of Nup50 fragments and truncations. FIG. 11B) Representative examples for the coexpression of mCherry-tagged Nup50 constructs and GFP-tagged TDP-43-mNLS. Scale bar=5 μm. FIG. 11C) Immunoblots of RIPA-insoluble fractions from HEK293 cells expressing mCherry-tagged Nup50 fragments and GFP-tagged TDP-mNLS and stained for TDP-43, mCherry, and β-actin as a loading control.

FIGS. 12A-12C. Nup50 activity towards insoluble TDP-43 lies in a short double alpha-helical domain. FIG. 12A) Schematic of Nup50 fragments and truncations. FIG. 12B) Immunoblots of RIPA-insoluble fractions from HEK293 cells expressing mCherry-tagged Nup50 fragments and GFP-tagged TDP-mNLS and stained for TDP-43, mCherry, and β-actin as a loading control. FIG. 12C) Representative examples for the coexpression of mCherry-tagged Nup50 constructs and GFP-tagged TDP-43-mNLS. Scale bar=5 μm. (on next slide).

FIGS. 13A-13B. Truncations removing 5 amino-acids (aa) from the N-terminus or C-terminus of the minimal active fragment aa160-200 eliminates effect on insoluble TDP-43-mNLS. FIG. 13A) Fluorescence microscopy showing representative examples for the coexpression of mCherry-tagged Nup50 constructs and GFP-tagged TDP-mNLS. Scale bar=5 μm. FIG. 13B) Immunoblots of RIPA-insoluble fractions from HEK293 cells expressing mCherry-tagged Nup50 fragments and GFP-tagged TDP-43-mNLS and stained for TDP-43, mCherry, and β-actin as a loading control.

FIGS. 14A-14E. A 41aa fragment of Nup50 polypeptide was sufficient to reduce pathological TDP-43 aggregation. FIG. 14A) Schematic of Nup50 fragments. FIG. 14B) Fluorescence microscopy showing representative examples for the coexpression of mCherry-tagged Nup50 constructs and GFP-tagged TDP-43-mNLS. Scale bar=5 μm. FIGS. 14C and 14D) Immunoblots of RIPA-insoluble fractions from HEK293 cells expressing mCherry-tagged Nup50 fragments and GFP-tagged TDP-43-mNLS and stained for TDP-43, mCherry, and β-actin as a loading control. FIG. 14E) AlphaFold predicted secondary structure of Nup50 protein with the 41aa smallest active fragment highlighted.

FIGS. 15A-15C. Further 5 amino acid truncation of the active 41aa Nup50 polypeptide abrogated its ability to reduce pathological TDP-43 aggregation. mCherry-tagged Nup50 fragments were co-expressed with GFP-TDP-43-mNLS in HEK293 cells and assessed via immunoblots of RIPA-insoluble fractions (FIG. 15A) and fluorescence microscopy (FIG. 15B). Immunoblots were stained for GFP and β-actin as a loading control. Scale bar=10 μm. Nuclear pore complex (NPC)-binding was assessed via fluorescence microscopy. Active, but not inactive, Nup50 fragments associated with the nuclear membrane (FIG. 15C).

FIGS. 16A-16C. Disrupting NPC-association abrogated Nup50 polypeptide activity towards TDP-43. FIG. 16A) Single amino acid changes in full-length human Nup50 were cloned and expressed in HEK293 cells and evaluated for NPC-binding. The Nup50 variant with decreased NPC-binding (Y190A), but not a benign neighboring variant, had decreased activity towards insoluble TDP-43-mNLS (FIGS. 16B and 16C).

FIGS. 17A-17D. Active Nup50-N polypeptide and aa160-200 polypeptide fragments interact with importins and nucleoporins. mCherry-tagged Nup50 fragments and an mCherry control were co-expressed with GFP-TDP-43-mNLS in HEK293 cells, immunoprecipitated (IP′d), and evaluated by immunoblotting and mass spectrometry. FIG. 17A) Active (Nup50-N and aa160-200) and inactive (aa160-195 and aa165-200) Nup50 fragments bind GFP-TDP-43-mNLS, but only active fragments bind Nup153. FIG. 17B) PCA plot showing IP-MS replicates clustered together. FIG. 17C (left)) Heatmap showing fragment binding to importins and nucleoporins. FIG. 17C (right)) Active, but not inactive fragments, bind to the nuclear pore and associate with the nuclear membrane via ICC. FIG. 17D) Quantification of Nup153 peptide frequency in IP-MS data.

FIGS. 18A-18C. Patient-derived mutations in a Nup50 polypeptide reduced Nup50 polypeptide ability to reduce pathological TDP-CTF aggregation. FIG. 18A) Schematic of Nup50 mutations. FIGS. 18B and 18C) mCherry-tagged Nup50 constructs with ALS-linked mutations were coexpressed in HEK293 cells with GFP-TDP-CTF and evaluated by immunoblots of RIPA-insoluble fractions. FIG. 18B) Immunoblots were probed for mCherry, GFP, and β-actin as a loading control. ALS-linked mutations D16fs, E20G, and F58fs decreased the ability of Nup50 to reduce insoluble TDP-CTF. FIG. 18C) Quantification of the immunoblot in FIG. 18B [is this accurate?].

FIGS. 19A-19C. Patient-derived mutations in a Nup50 polypeptide reduced Nup50 polypeptide ability to reduce pathological TDP-CTF aggregation. mCherry-tagged Nup50 constructs with ALS-linked mutations were coexpressed in HEK293 cells with GFP-TDP-43-mNLS and evaluated by immunoblots of RIPA-insoluble fractions. FIG. 19A) Immunoblots were probed for mCherry, GFP, and β-actin as a loading control. ALS-linked mutations D16fs, E20G, and F58fs decreased the ability of Nup50 to reduce insoluble TDP-43-mNLS. Patient-derived Nup50 variants were assessed for NPC-binding. FIG. 19B) Quantification of the immunoblot in FIG. 19A [is this accurate?]. FIG. 19C) Nup50-D16fs and Nup50-F58fs had diminished binding to the NPC and association with the nuclear membrane.

FIG. 20. Nup50 polypeptide expression reduces TDP-CTF aggregation in mouse organotypic brain slice cultures. GFP-TDP-CTF and mScarlet or mScarlet-Nup50-L were co-expressed in mouse organotypic brain slice cultures for 14 days and assessed via fluorescence microscopy. Nup50-L expression decreased number and size of TDP-CTF aggregates.

### DETAILED DESCRIPTION

This document provides methods and materials for treating a mammal (e.g., a human) having, or at risk of developing, a proteinopathy (e.g., a TDP-43 proteinopathy). For example, one or more nucleoporin polypeptides and/or fragments thereof (and/or nucleic acids designed to express a nucleoporin polypeptide and/or a fragment thereof) can be administered to a mammal (e.g., a human) having, or at risk of developing, a proteinopathy (e.g., a TDP-43 proteinopathy) to treat the mammal.

In some cases, the methods and materials described herein can be used to treat a proteinopathy (e.g., a TDP-43 proteinopathy). For example, one or more nucleoporin polypeptides (and/or one or more nucleic acids designed to express a nucleoporin polypeptide) can be administered to a mammal (e.g., a human) in need thereof (e.g., a human having, or at risk of developing, a proteinopathy such as a TDP-43 proteinopathy) to slow, delay, or prevent progression of a proteinopathy (e.g., a TDP-43 proteinopathy). In some cases, one or more nucleoporin polypeptides (and/or one or more nucleic acids designed to express a nucleoporin polypeptide) can be administered to a mammal (e.g., a human) in need thereof (e.g., a human having, or at risk of developing, a proteinopathy such as a TDP-43 proteinopathy) to slow, delay, or prevent the development of a proteinopathy (e.g., a TDP-43 proteinopathy).

In some cases, the methods and materials described herein can be used to reduce or eliminate one or more symptoms of a proteinopathy (e.g., a TDP-43 proteinopathy). For example, one or more nucleoporin polypeptides (and/or one or more nucleic acids designed to express a nucleoporin polypeptide) can be administered to a mammal (e.g., a human) in need thereof (e.g., a human having, or at risk of developing, a proteinopathy such as a TDP-43 proteinopathy) to reduce or eliminate one or more symptoms of a proteinopathy (e.g., a TDP-43 proteinopathy). Examples of symptoms of a proteinopathy (e.g., a TDP-43 proteinopathy) include, without limitation, deterioration in behavior and personality (e.g., affecting conduct, judgment, empathy, and foresight), depression, apathy, social withdrawal, mood swings, irritability, aggressiveness, changes in sleeping habits, wandering, loss of inhibitions, delusions, emotional blunting, compulsive or ritualistic behavior, changes in eating habits or diet, deficits in executive function, lack of insight, agitation, emotional instability, difficulty producing or comprehending spoken or written language, memory loss, and symptoms of motor neuron disease such as muscle weakness, muscle atrophy, fasciculations, spasticity, dysarthria, and dysphagia. In some cases, the materials and methods described herein can be used to reduce the severity of one or more symptoms of a proteinopathy (e.g., a TDP-43 proteinopathy) in a mammal (e.g., a human) by, for example, 10, 20, 30, 40, 50, 60, 70, 80, 90, 95, or more percent.

In some cases, the methods and materials described herein can be used to reduce or eliminate neurodegeneration associated with a proteinopathy (e.g., a TDP-43 proteinopathy). For example, one or more nucleoporin polypeptides (and/or one or more nucleic acids designed to express a nucleoporin polypeptide) can be administered to a mammal (e.g., a human) in need thereof (e.g., a human having, or at risk of developing, a proteinopathy such as a TDP-43 proteinopathy) to slow, delay, or prevent progression of neurodegeneration associated with a proteinopathy (e.g., a TDP-43 proteinopathy). In some cases, one or more nucleoporin polypeptides (and/or one or more nucleic acids designed to express a nucleoporin polypeptide) can be administered to a mammal (e.g., a human) in need thereof (e.g., a human having, or at risk of developing, a proteinopathy such as a TDP-43 proteinopathy) to slow, delay, or prevent the development of neurodegeneration associated with a proteinopathy (e.g., a TDP-43 proteinopathy).

In some cases, the methods and materials described herein can be used to reduce or eliminate aggregation of TDP-43 polypeptides. For example, the materials and methods described herein can be used to reduce the number of TDP-43 polypeptide aggregates present within the central nervous system (CNS; e.g., within the brain and/or spinal cord) of a mammal having a proteinopathy (e.g., a TDP-43 proteinopathy) by, for example, 10, 20, 30, 40, 50, 60, 70, 80, 90, 95, or more percent. For example, the materials and methods described herein can be used to reduce the size (e.g., volume) of one or more TDP-43 polypeptide aggregates present within the CNS (e.g., within the brain and/or spinal cord) of a mammal having a proteinopathy (e.g., a TDP-43 proteinopathy) by, for example, 10, 20, 30, 40, 50, 60, 70, 80, 90, 95, or more percent. For example, the materials and methods described herein can be used to reduce the fraction of detergent-insoluble TDP-43 polypeptides present within the CNS (e.g., within the brain and/or spinal cord) of a mammal having a proteinopathy (e.g., a TDP-43 proteinopathy) by, for example, 10, 20, 30, 40, 50, 60, 70, 80, 90, 95, or more percent.

In some cases, the methods and materials described herein can be used to reduce or eliminate cell death associated with a proteinopathy (e.g., a TDP-43 proteinopathy). For example, the materials and methods described herein can be used to reduce the number of apoptotic cells present within the CNS (e.g., within the brain and/or spinal cord) of a mammal having a proteinopathy (e.g., a TDP-43 proteinopathy) by, for example, 10, 20, 30, 40, 50, 60, 70, 80, 90, 95, or more percent.

In some cases, the methods and materials described herein can be used to restore nuclear localization of TDP-43 polypeptides. For example, the materials and methods described herein can be used to increase the number of TDP-43 polypeptides that can localize the nucleus of cells present within the CNS (e.g., within the brain and/or spinal cord) of a mammal having a proteinopathy (e.g., a TDP-43 proteinopathy) by, for example, 10, 20, 30, 40, 50, 60, 70, 80, 90, 95, or more percent.

In some cases, the methods and materials described herein can be used to restore the cellular function of TDP-43 polypeptides. For example, the materials and methods described herein can be used to increase a level of one or more polypeptides whose expression is regulated by TDP-43 polypeptides in cells present within the CNS of a mammal having a proteinopathy (e.g., a TDP-43 proteinopathy) by, for example, 10, 20, 30, 40, 50, 60, 70, 80, 90, 95, or more percent. For example, the materials and methods described herein can be used to increase splicing and/or poly-adenylation of a nucleic acid (e.g., an mRNA) encoding a polypeptide whose expression is regulated by TDP-43 polypeptides in cells present within the CNS of a mammal having a proteinopathy (e.g., a TDP-43 proteinopathy) by, for example, 10, 20, 30, 40, 50, 60, 70, 80, 90, 95, or more percent. Examples of polypeptides whose expression is regulated by TDP-43 polypeptides include, without limitation, Stathmin-2 polypeptides and UNC13A polypeptides.

Any appropriate mammal having, or at risk of developing, a proteinopathy (e.g., a TDP-43 proteinopathy) can be treated as described herein (e.g., by administering one or more nucleoporin polypeptides and/or fragments thereof and/or nucleic acids designed to express a nucleoporin polypeptide and/or a fragment thereof). Examples of mammals that can be treated as described herein include, without limitation, humans, non-human primates (e.g., monkeys), dogs, cats, horses, camels, cows, pigs, sheep, mice, rats, rabbits, hamster, guinea pigs, and ferrets.

When treating a mammal (e.g., a human) having, or at risk of developing, a proteinopathy (e.g., a TDP-43 proteinopathy) as described herein (e.g., by administering one or more nucleoporin polypeptides and/or fragments thereof and/or nucleic acids designed to express a nucleoporin polypeptide and/or a fragment thereof), the proteinopathy can be any type of proteinopathy. In some cases, a proteinopathy can be a TDP-43 proteinopathy (e.g., can include aggregation of TDP-43 polypeptides). In some cases, a proteinopathy can be a neurodegenerative disease. Examples of types of proteinopathies that can be treated as described herein include, without limitation, FTD, ALS, Alzheimer's disease, traumatic brain injury (TBI), chronic traumatic encephalopathy (CTE), limbic-predominant age-related TDP-43 encephalopathy (LATE), dementia with Lewy bodies (DLB), Parkinson's disease, Huntington's disease, argyrophilic grain disease (AGD), hippocampal sclerosis (HS), Guam ALS, Guam parkinsonism-dementia complex (G-PDC), Perry disease, facial onset sensory and motor neuronopathy (FOSMN), inclusion body myositis (IBM), oculopharyngeal muscular dystrophy (OPMD), and distal myopathies with rimmed vacuoles (DMRV).

In some cases, a mammal (e.g., a human) can be identified as having, or as being at risk of developing, a proteinopathy (e.g., a TDP-43 proteinopathy). For example, genetic testing, imaging techniques (e.g., brain scanning techniques such as magnetic resonance imaging (MRI), computed tomography (CT) scanning, fluorodeoxyglucose positron emission tomography (FDG-PET) scanning, and SPECT (single proton emission CT) scanning)), and laboratory techniques (e.g., to test samples for biomarkers) such as enzyme-linked immunosorbent assay (ELISA), immunohistochemistry (IHC), cerebrospinal fluid real-time quaking-induced conversion (RT-QuIC), single molecule array (Simoa), proximity extension assay (PEA), western blot analysis, and/or mass spectrometry can be used to identify a mammal as having, or as being at risk of developing, a proteinopathy (e.g., a TDP-43 proteinopathy).

Once identified as having, or as being at risk of developing, a proteinopathy (e.g., a TDP-43 proteinopathy), the mammal (e.g., the human) can be administered, or instructed to self-administer, one or more nucleoporin polypeptides and/or fragments thereof (and/or nucleic acids designed to express a nucleoporin polypeptide and/or a fragment thereof) as described herein.

Any appropriate nucleoporin polypeptide or fragment thereof (and/or nucleic acid designed to express a nucleoporin polypeptide or a fragment thereof) can be administered to a mammal (e.g., a human) having, or at risk of developing, a proteinopathy (e.g., a TDP-43 proteinopathy) as described herein. Examples of nucleoporin polypeptides include, without limitation, Nup50 polypeptides and Nup98 polypeptides.

When a nucleoporin polypeptide is a Nup50 polypeptide, any appropriate Nup50 polypeptide (and/or nucleic acid designed to express a Nup50 polypeptide) can be administered to a mammal (e.g., a human) having, or at risk of developing, a proteinopathy (e.g., a TDP-43 proteinopathy) as described herein. Examples of Nup50 polypeptides and nucleic acids encoding Nup50 polypeptides include, without limitation, those set forth in the National Center for Biotechnology Information (NCBI) databases at, for example, accession no. NM_007172 (version NM_007172.4), accession no. NM_153645 (version NM_153645.2), accession no. NP_009103 (version NP_009103.2), and accession no. NP_705931 (version NP_705931.1).

In some cases, a nucleic acid encoding a Nup50 polypeptide can have a nucleotide sequence set forth in SEQ ID NO:1 or SEQ ID NO:2 (see, e.g., Example 1). In some cases, a Nup50 polypeptide can have an amino acid sequence set forth in SEQ ID NO:3 or SEQ ID NO:4 (see, e.g., Example 2). In some cases, a Nup50 polypeptide can have an amino acid sequence set forth in any one of SEQ ID NOs: 14-24 (see, e.g., Example 2).

When a fragment of a nucleoporin polypeptide is a Nup50 polypeptide fragment, any appropriate Nup50 polypeptide fragment (and/or nucleic acid designed to express a Nup50 polypeptide fragment) can be administered to a mammal (e.g., a human) having, or at risk of developing, a proteinopathy (e.g., a TDP-43 proteinopathy) as described herein. In some cases, a Nup50 polypeptide fragment can be derived from the amino acid sequence set forth in SEQ ID NO:3. For example, a Nup50 polypeptide fragment can be derived from the amino acid sequence of SEQ ID NO:3, provided that it maintains at least some function of a full-length Nup50 polypeptide (e.g., the ability to reduce at least some TDP-43 aggregation, restore TDP-43 nuclear localization, and/or reduce TDP-43 cellular toxicity). In some cases, a Nup50 polypeptide fragment can be derived from the amino acid sequence set forth in SEQ ID NO:4. For example, a Nup50 polypeptide fragment can be derived from the amino acid sequence of SEQ ID NO:4, provided that it maintains at least some function of a full-length Nup50 polypeptide (e.g., the ability to reduce at least some TDP-43 aggregation, restore TDP-43 nuclear localization, and/or reduce TDP-43 cellular toxicity). In some cases, a Nup50 polypeptide fragment can be derived from the amino acid sequence set forth in any one of SEQ ID NOs:14-24. For example, a Nup50 polypeptide fragment can be derived from the amino acid sequence of any one of SEQ ID NOs:14-24, provided that it maintains at least some function of a full-length Nup50 polypeptide (e.g., the ability to reduce at least some TDP-43 aggregation, restore TDP-43 nuclear localization, and/or reduce TDP-43 cellular toxicity).

ANup50 polypeptide fragment can be any appropriate length (e.g., can include any number of amino acids). In some cases, a Nup50 polypeptide fragment can be from about 35 amino acids in length to about 470 amino acids in length (e.g., from about 35 amino acids in length to about 450 amino acids, from about 35 amino acids in length to about 400 amino acids, from about 35 amino acids in length to about 350 amino acids, from about 35 amino acids in length to about 300 amino acids, from about 35 amino acids in length to about 250 amino acids, from about 35 amino acids in length to about 200 amino acids, from about 35 amino acids in length to about 150 amino acids, from about 35 amino acids in length to about 100 amino acids, from about 35 amino acids in length to about 75 amino acids, from about 35 amino acids in length to about 50 amino acids, from about 50 amino acids in length to about 470 amino acids, from about 100 amino acids in length to about 470 amino acids, from about 150 amino acids in length to about 470 amino acids, from about 200 amino acids in length to about 470 amino acids, from about 250 amino acids in length to about 470 amino acids, from about 300 amino acids in length to about 470 amino acids, from about 350 amino acids in length to about 470 amino acids, from about 400 amino acids in length to about 470 amino acids, from about 50 amino acids in length to about 450 amino acids, from about 75 amino acids in length to about 400 amino acids, from about 100 amino acids in length to about 350 amino acids, from about 150 amino acids in length to about 300 amino acids, from about 200 amino acids in length to about 250 amino acids, from about 50 amino acids in length to about 100 amino acids, from about 100 amino acids in length to about 150 amino acids, from about 150 amino acids in length to about 200 amino acids, from about 200 amino acids in length to about 250 amino acids, from about 250 amino acids in length to about 300 amino acids, from about 300 amino acids in length to about 350 amino acids, from about 350 amino acids in length to about 400 amino acids, or from about 400 amino acids in length to about 450 amino acids in length) provided that it maintains at least some function of a full-length Nup50 polypeptide (e.g., the ability to reduce at least some TDP-43 aggregation). For example, a Nup50 polypeptide fragment can be about 40 amino acids in length.

In some cases, a Nup50 polypeptide can comprise, consist essentially of, or consist of the amino acid sequence set forth in SEQ ID NO:3 or SEQ ID NO:4. In some cases, a Nup50 polypeptide can comprise, consist essentially of, or consist of the amino acid sequence set forth in any one of SEQ ID NOs:14-24. In some cases, a Nup50 polypeptide fragment can comprise, consist essentially of, or consist of an amino acid sequence set forth in Table 1.

TABLE 1
Exemplary Nup50 polypeptide fragments.

| | | SEQ ID |
|---|---|---|
| Truncation | Polypeptide Sequence | NO: |
| Nup50-S | MASEEVLKNRAIKKAKRRNVGFESDTGGAFKGFKGLVVPSGGG | 5 |
| (29-468) | RFSGFGSGAGGKPLEGLSNGNNITSAPPFASAKAAADPKVAFG | |
| | SLAANGPTTLVDKVSNPKINGDSQQPSSSGLASSKACVGNAYH | |
| | KQLAALNCSVRDWIVKHVNTNPLCDLTPIFKDYEKYLANIEQQ | |
| | HGNSGRNSESESNKVAAETQSPSLEGSTKLQQESTFLFHGNKT | |
| | EDTPDKKMEVASEKKTDPSSLGATSASFNFGKKVDSSVLGSLS | |
| | SVPLTGFSFSPGNSSLFGKDTTQSKPVSSPFPTKPLEGQAEGD | |
| | SGECKGGDEEENDEPPKVVVTEVKEEDAFYSKKCKLFYKKDNE | |
| | FKEKGIGTLHLKPTANQKTQLLVRADTNLGNILLNVLIPPNMP | |
| | CTRTGKNNVLIVCVPNPPIDEKNATMPVTMLIRVKTSEDADEL | |
| | HKILLEKKDA | |
| Nup50-N | MAKRNAEKELTDRNWDQEDEAEDVGTFSMASEEVLKNRAIKKA | 6 |
| (1-210) | KRRNVGFESDTGGAFKGFKGLVVPSGGGRESGFGSGAGGKPLE | |
| | GLSNGNNITSAPPFASAKAAADPKVAFGSLAANGPTTLVDKVS | |
| | NPKTNGDSQQPSSSGLASSKACVGNAYHKQLAALNCSVRDWIV | |
| | KHVNTNPLCDLTPIFKDYEKYLANIEQQHGNSGRNSES | |
| Nup50 47-210 | NVGFESDTGGAFKGFKGLVVPSGGGRFSGFGSGAGGKPLEGLS | 7 |
| | NGNNITSAPPFASAKAAADPKVAFGSLAANGPTTLVDKVSNPK | |
| | TNGDSQQPSSSGLASSKACVGNAYHKQLAALNCSVRDWIVKHV | |
| | NTNPLCDLTPIFKDYEKYLANIEQQHGNSGRNSES | |
| Nup50 150-210 | ACVGNAYHKQLAALNCSVRDWIVKHVNTNPLCDLTPIFKDYEK | 8 |
| | YLANIEQQHGNSGRNSES | |
| Nup50 160-210 | LAALNCSVRDWIVKHVNTNPLCDLTPIFKDYEKYLANIEQQHG | 9 |
| | NSGRNSES | |
| Nup50 150-200 | ACVGNAYHKQLAALNCSVRDWIVKHVNTNPLCDLTPIFKDYEK | 10 |
| | YLANIEQQ | |
| Nup50 160-200 | LAALNCSVRDWIVKHVNTNPLCDLTPIFKDYEKYLANIEQQ | 11 |
| Nup50-D16fs | MAKRNAEKELTDRNWIGIGIKKMKLKMWEHSPWPVRKS | 12 |
| Nup50-F58fs | MAKRNAEKELTDRNWDQEDEAEDVGTFSMASEEVLKNRAIKKA | 13 |
| | KRRNVGFESDTGGALKVLKVWWYLLEEDAFLDLVVALEGSLWK | |
| | DCRMETT | |

In some cases, a Nup50 polypeptide fragment provided herein can include the amino acid sequence set forth in any one of SEQ ID NOs:5-13 with zero, one, or two amino acid substitutions within the articulated sequence of the sequence identifier (e.g., any one of SEQ ID NOs:5-13), with zero, one, two, three, four, or five amino acid residues preceding the articulated sequence of the sequence identifier (e.g., any one of SEQ ID NOs:5-13), and/or with zero, one, two, three, four, or five amino acid residues following the articulated sequence of the sequence identifier (e.g., any one of SEQ ID NOs:5-13), provided that the Nup50 polypeptide fragment retains at least some activity exhibited by a full-length Nup50 polypeptide (e.g., the ability to reduce at least some TDP-43 aggregation, restore TDP-43 nuclear localization, and/or reduce TDP-43 cellular toxicity).

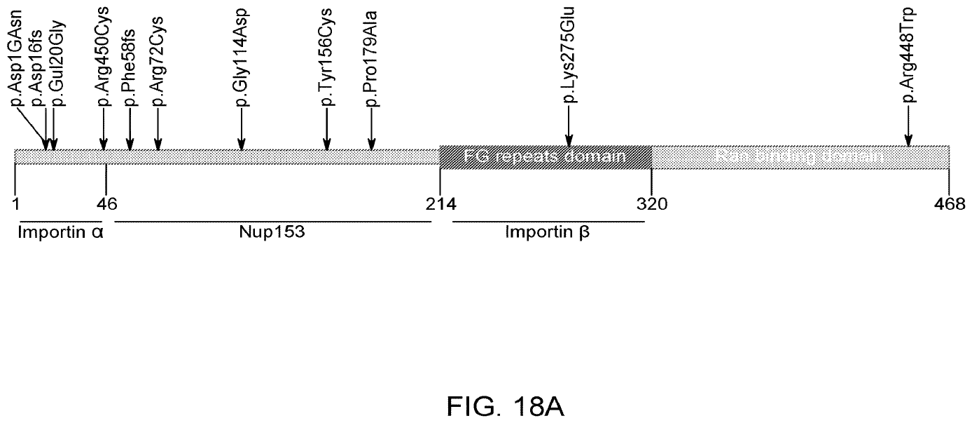

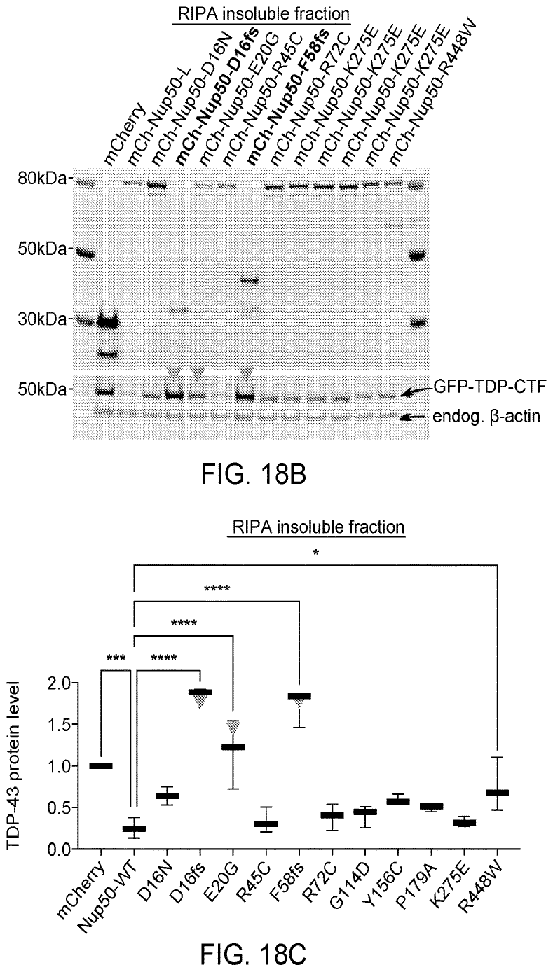

In some cases, a Nup50 polypeptide fragment provided herein can include one or more modified amino acid residues. For example, a Nup50 polypeptide fragment provided herein can include one or more nonnatural amino acids. In some cases, a modified amino acid that can be present in a Nup50 polypeptide fragment provided herein can be as shown in FIG. 18. In some cases, a modified amino acid that can be present in a Nup50 polypeptide fragment provided herein can be as described elsewhere (see, e.g., Megat et al., Nat. Commun., 14:342 (2023)).

In some cases, a Nup50 polypeptide fragment provided herein can be in the form of a peptide analog (e.g., a peptide analog that can mimic the structural elements and/or activity of a Nup50 polypeptide fragment provided herein). For example, a Nup50 polypeptide fragment provided herein can be a peptidomimetic.

Any appropriate method can be used to deliver one or more nucleoporin polypeptides or fragments thereof (and/or nucleic acids designed to express a nucleoporin polypeptide or a fragment thereof) to a mammal. When one or more nucleoporin polypeptides or fragments thereof (and/or nucleic acids designed to express a nucleoporin polypeptide or a fragment thereof) are administered to a mammal (e.g., a human), the one or more nucleoporin polypeptides or fragments thereof (and/or nucleic acids designed to express a nucleoporin polypeptide or a fragment thereof) can be administered to the CNS (e.g., within the brain and/or spinal cord) of a mammal (e.g., a human). In some cases, one or more nucleoporin polypeptides or fragments thereof (and/or nucleic acids designed to express a nucleoporin polypeptide or a fragment thereof) can be administered to the CNS (e.g., within the brain and/or spinal cord) of a mammal (e.g., a human) by direct injection into the CNS.

Any appropriate method can be used to obtain a nucleoporin polypeptide or fragment thereof provided herein (e.g., a polypeptide that comprises, consists essentially of, or consists of the amino acid sequence set forth in any one of SEQ ID NOs:3-24). For example, a nucleoporin polypeptide or fragment thereof provided herein (e.g., a polypeptide that comprises, consists essentially of, or consists of the amino acid sequence set forth in any one of SEQ ID NOs:3-24) can be obtained by synthesizing the polypeptide of interest using appropriate polypeptide synthesizing techniques.

When one or more nucleic acids designed to express a nucleoporin polypeptide or a fragment thereof are administered to a mammal (e.g., a human), the nucleic acid can be in the form of a vector (e.g., a viral vector or a non-viral vector).

When a vector used to deliver nucleic acid encoding a nucleoporin polypeptide or a fragment thereof to a mammal (e.g., a human) is a viral vector, any appropriate viral vector can be used. A viral vector can be derived from a positive-strand virus or a negative-strand virus. A viral vector can be derived from a virus with a DNA genome or an RNA genome. In some cases, a viral vector can be a chimeric viral vector. In some cases, a viral vector can infect dividing cells. In some cases, a viral vector designed to deliver nucleic acid encoding a nucleoporin polypeptide (or fragment thereof) can infect non-dividing cells. Examples virus-based vectors that can be used to deliver nucleic acid encoding a nucleoporin polypeptide to a mammal (e.g., a human) include, without limitation, virus-based vectors based on adenoviruses, AAVs, Sendai viruses, retroviruses, lentiviruses, herpes simplex viruses (HSV), vaccinia viruses, or baculoviruses.

When a vector used to deliver nucleic acid encoding a nucleoporin polypeptide or a fragment thereof to a mammal (e.g., a human) is a non-viral vector, any appropriate non-viral vector can be used. In some cases, a non-viral vector can be an expression plasmid (e.g., a cDNA expression vector).

When one or more nucleic acids designed to express a nucleoporin polypeptide or a fragment thereof are administered to a mammal (e.g., a human), the nucleic acid can be included in (e.g., encapsulated within) a carrier molecule. For example, one or more nucleic acids designed to express a nucleoporin polypeptide or a fragment thereof can be included in (e.g., encapsulated within) a nanoparticle (e.g., a lipid nanoparticle), and one or more of the nanoparticles can be administered to a mammal (e.g., a human).

When nucleic acid encoding a nucleoporin polypeptide or a fragment thereof is administered to a mammal, the nucleic acid can be used for transient expression of a nucleoporin polypeptide or a fragment thereof or for stable expression of a nucleoporin polypeptide or a fragment thereof. In cases where a nucleic acid encoding a nucleoporin polypeptide or a fragment thereof is used for stable expression of a nucleoporin polypeptide or a fragment thereof, the nucleic acid encoding a nucleoporin polypeptide or a fragment thereof can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acid designed to express a nucleoporin polypeptide or a fragment thereof into the genome of a cell.

In addition to nucleic acid encoding a nucleoporin polypeptide or a fragment thereof, a vector (e.g., a viral vector or a non-viral vector) can contain one or more regulatory elements operably linked to the nucleic acid encoding a nucleoporin polypeptide or a fragment thereof. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a nucleoporin polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner (e.g., cytomegalovirus/chicken beta-actin (CBA) hybrid promoters, cytomegalovirus (CMV) early enhancer/promoters, ubiquitin C (UbC) promoters, prion protein (Prp) promoters, calcium/calmodulin-dependent protein Kinase II (CaMKII) promoters, synapsin I (SYN) promoters, methyl-CpG-binding protein-2 (MeCP2) promoters, neuron-specific enolase (NSE) promoters, vesicular glutamate transporter promoter (vGLUT) promoters, and Hb9 promoters). Examples of promoters that can be used to drive expression of a nucleoporin polypeptide in cells include, without limitation, Prp promoters, CaMKII promoters, SYN promoters, MeCP2 promoters, NSE promoters, vGLUT promoters, and Hb9 promoters. As used herein, “operably linked” refers to positioning of a regulatory element relative to a nucleic acid encoding a polypeptide in such a way as to permit or facilitate expression of the encoded polypeptide. For example, a vector can contain a promoter and nucleic acid encoding a nucleoporin polypeptide or a fragment thereof. In this case, the promoter is operably linked to a nucleic acid encoding a nucleoporin polypeptide or a fragment thereof such that it drives expression of the nucleoporin polypeptide or a fragment thereof in cells. In some cases, nucleic acid encoding a nucleoporin polypeptide or a fragment thereof can contain nucleic acid encoding a detectable label. For example, a vector can include nucleic acid encoding a nucleoporin polypeptide or a fragment thereof and nucleic acid encoding a detectable label positioned such that the encoded polypeptide is a fusion polypeptide that includes a nucleoporin polypeptide or a fragment thereof fused to a detectable polypeptide. In some cases, a detectable label can be a peptide tag. In some cases, a detectable label can be a fluorescent molecule (e.g., fluorescent polypeptides). Examples of detectable labels that can be used as described herein include, without limitation, an HA tag, a Myc-tag, a FLAG-tag, green fluorescent polypeptides (GFPs; e.g., enhanced GFPs), red fluorescent polypeptides (e.g. mCherry polypeptides), Halo tags, SNAP-tags, 6xHis-tags, GST-tags, MBP-tags, strep-tags, and V5-tags.

Nucleic acid encoding a nucleoporin polypeptide or a fragment thereof can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a nucleoporin polypeptide.

In some cases, one or more nucleoporin polypeptides or fragments thereof (and/or nucleic acids designed to express a nucleoporin polypeptide or a fragment thereof) can be formulated into a composition (e.g., a pharmaceutical composition) for administration to a mammal (e.g., a human). For example, one or more nucleoporin polypeptides or fragments thereof (and/or nucleic acids designed to express a nucleoporin polypeptide or a fragment thereof) can be formulated into a pharmaceutically acceptable composition for administration to a mammal (e.g., a human) having, or at risk of developing, a proteinopathy (e.g., a TDP-43 proteinopathy). In some cases, one or more nucleoporin polypeptides or fragments thereof (and/or nucleic acids designed to express a nucleoporin polypeptide or a fragment thereof) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition (e.g., a pharmaceutical composition) containing one or more nucleoporin polypeptides or fragments thereof (and/or nucleic acids designed to express a nucleoporin polypeptide or a fragment thereof) can be formulated into any appropriate dosage form. Examples of dosage forms include solid or liquid forms including, without limitation, gels, liquids, suspensions, solutions (e.g., sterile solutions), sustained-release formulations, and delayed-release formulations.

A composition (e.g., a pharmaceutical composition) containing one or more nucleoporin polypeptides or fragments thereof (and/or nucleic acids designed to express a nucleoporin polypeptide or a fragment thereof) can be designed for parenteral (e.g., intracerebral injections, intracerebroventricular (ICV) injections, intra cisterna magna (ICM) injections, intrathecal (IT) injections, intraparenchymal injections, intramuscular injections, and intranasal delivery) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more nucleoporin polypeptides or fragments thereof (and/or nucleic acids designed to express a nucleoporin polypeptide or a fragment thereof) can be administered locally or systemically. For example, a composition containing one or more nucleoporin polypeptides or fragments thereof (and/or nucleic acids designed to express a nucleoporin polypeptide or a fragment thereof) can be administered locally by direct injection (e.g., an intracerebral injection) to the CNS (e.g., within the brain and/or spinal cord) of a mammal (e.g., a human).

An effective amount of a composition (e.g., a pharmaceutical composition) containing one or more nucleoporin polypeptides or fragments thereof (and/or nucleic acids designed to express a nucleoporin polypeptide or a fragment thereof) can be any amount that can treat the mammal without producing significant toxicity to the mammal. For example, an effective amount of one or more nucleoporin polypeptides or fragments thereof can be from about 0.1 milligrams of polypeptide(s) per kilogram bodyweight of the mammal (mg/kg) per dose to about 100 mg/kg per dose (e.g., from about 0.1 mg/kg to about 80 mg/kg, from about 0.1 mg/kg to about 60 mg/kg, from about 0.1 mg/kg to about 50 mg/kg, from about 0.1 mg/kg to about 40 mg/kg, from about 0.1 mg/kg to about 30 mg/kg, from about 0.1 mg/kg to about 20 mg/kg, from about 0.1 mg/kg to about 10 mg/kg, from about 1 mg/kg to about 100 mg/kg, from about 10 mg/kg to about 100 mg/kg, from about 25 mg/kg to about 100 mg/kg, from about 50 mg/kg to about 100 mg/kg, from about 75 mg/kg to about 100 mg/kg, from about 1 mg/kg to about 75 mg/kg, from about 10 mg/kg to about 50 mg/kg, from about 20 mg/kg to about 40 mg/kg, from about 1 mg/kg to about 20 mg/kg, from about 20 mg/kg to about 40 mg/kg, from about 40 mg/kg to about 60 mg/kg, or from about 60 mg/kg to about 80 mg/kg per dose). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a composition (e.g., a pharmaceutical composition) containing one or more nucleoporin polypeptides or fragments thereof (and/or nucleic acids designed to express a nucleoporin polypeptide or a fragment thereof) can be any frequency that can treat a mammal (e.g., a human) having, or at risk of developing, a proteinopathy (e.g., a TDP-43 proteinopathy) without producing significant toxicity to the mammal. For example, the frequency of administration can be from about two times a day to about once a month, from about once a day to about twice a month, or from about once a week to about every two weeks. The frequency of administration can remain constant or can be variable during the duration of treatment. A course of treatment with a composition containing one or more nucleoporin polypeptides or fragments thereof (and/or nucleic acids designed to express a nucleoporin polypeptide or a fragment thereof) can include rest periods. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition (e.g., a pharmaceutical composition) containing one or more nucleoporin polypeptides or fragments thereof (and/or nucleic acids designed to express a nucleoporin polypeptide or a fragment thereof) can be any duration that treat a mammal (e.g., a human) having, or at risk of developing, a proteinopathy (e.g., a TDP-43 proteinopathy) without producing significant toxicity to the mammal. For example, the effective duration can vary from several days to several weeks, months, or years. In some cases, the effective duration for the treatment of a mammal (e.g., a human) having, or at risk of developing, a proteinopathy (e.g., a TDP-43 proteinopathy) can be the duration of the life of the mammal. Multiple factors can influence the actual effective duration used for a particular treatment. For example, an effective duration can vary with the frequency of administration, effective amount, use of multiple treatment agents, route of administration, and severity of the condition being treated.

In some cases, the one or more nucleoporin polypeptides or fragments thereof(and/or nucleic acids designed to express a nucleoporin polypeptide or a fragment thereof) can be used as the sole active agent used to treat a mammal (e.g., a human) having, or at risk of developing, a proteinopathy (e.g., a TDP-43 proteinopathy).

In some cases, the methods and materials described herein can include one or more (e.g., one, two, three, four, five or more) additional agents used to treat a mammal (e.g., a human) having, or at risk of developing, a proteinopathy (e.g., a TDP-43 proteinopathy). In some cases, an agent used to treat a mammal having, or at risk of developing, a proteinopathy can be a small molecule. In some cases, an agent used to treat a mammal having, or at risk of developing, a proteinopathy can be an anti-sense oligonucleotide (ASO). In some cases, an agent used to treat a mammal having, or at risk of developing, a proteinopathy can be a polypeptide (e.g., an antibody). Examples of agents used to treat a proteinopathy (e.g., a TDP-43 proteinopathy) that can be administered to a mammal (e.g., a human) having, or at risk of developing, a proteinopathy (e.g., a TDP-43 proteinopathy) together with one or more nucleoporin polypeptides or fragments thereof (and/or nucleic acids designed to express a nucleoporin polypeptide or a fragment thereof) include, without limitation, antidepressants, antipsychotics, riluzole (e.g., RILUTEK®), edavarone (e.g., RADICAVA ORSn), agents that can target mutant superoxide dismutase 1 (SOD1) polypeptides, agents that can target C9orf72 repeat expansions, and agents that can target amyloid beta (Ap) polypeptides. In some cases, the one or more additional agents can be administered together with one or more nucleoporin polypeptides or fragments thereof (and/or nucleic acids designed to express a nucleoporin polypeptide or a fragment thereof, e.g., in the same composition). In some cases, the one or more additional agents can be administered independent of the one or more nucleoporin polypeptides or fragments thereof (and/or nucleic acids designed to express a nucleoporin polypeptide or a fragment thereof). When the one or more additional agents are administered independent of the one or more nucleoporin polypeptides or fragments thereof (and/or nucleic acids designed to express a nucleoporin polypeptide or a fragment thereof), the one or more nucleoporin polypeptides or fragments thereof (and/or nucleic acids designed to express a nucleoporin polypeptide or a fragment thereof) can be administered first, and the one or more additional agents administered second, or vice versa.

In some cases, the methods and materials described herein can include subjecting a mammal having, or at risk of developing, a proteinopathy (e.g., a TDP-43 proteinopathy) to one or more (e.g., one, two, three, four, five or more) additional therapies (e.g., therapeutic interventions) that are effective to treat a proteinopathy (e.g., a TDP-43 proteinopathy). Examples of therapies that can be used to treat a proteinopathy include, without limitation, physical therapy, occupational therapy, speech therapy, and any combinations thereof. In some cases, the one or more additional treatments that are effective to treat a proteinopathy (e.g., a TDP-43 proteinopathy) can be performed at the same time as the administration of the one or more nucleoporin polypeptides or fragments thereof (and/or nucleic acids designed to express a nucleoporin polypeptide or a fragment thereof). In some cases, the one or more additional treatments that are effective to treat a proteinopathy (e.g., a TDP-43 proteinopathy) can be performed before and/or after the administration of the one or more nucleoporin polypeptides or fragments thereof (and/or nucleic acids designed to express a nucleoporin polypeptide or a fragment thereof).

The invention will be further described in the following examples, which do not limit the scope of the invention described in the claims.

### EXAMPLES

### Example 1: Exemplary Nucleic Acid Sequences Encoding a Nup50 Polypeptide

Nucleic Acid Encoding an Exemplary Nup50 Polypeptide:
(SEQ ID NO: 1)
ATGGCCAGTGAGGAAGTCTTGAAGAATAGAGCCATAAAGAAAGCAAAGCGCAGAAATGTTGG
ATTTGAATCTGACACTGGAGGAGCCTTTAAAGGTTTTAAAGGTTTGGTGGTACCTTCTGGAG
GAGGACGCTTTTCTGGATTTGGTAGTGGCGCTGGAGGGAAGCCTTTGGAAGGACTGTCGAAT
GGAAACAACATAACCAGTGCCCCTCCCTTCGCCAGTGCAAAGGCAGCGGCAGATCCCAAGGT
AGCCTTTGGTTCTCTTGCTGCAAATGGCCCTACCACCTTGGTTGATAAAGTTTCAAATCCCA
AAACTAATGGGGACAGTCAGCAGCCCTCCTCCTCTGGCCTTGCTTCCAGTAAAGCTTGTGTC
GGAAATGCCTATCACAAGCAGTTGGCCGCCTTGAACTGCTCCGTGCGGGATTGGATAGTGAA
GCACGTGAATACAAACCCCCTCTGTGATCTGACACCTATCTTTAAAGACTATGAGAAATATT
TAGCAAACATTGAACAGCAACACGGGAACAGTGGCAGGAATTCTGAAAGTGAATCTAACAAA
GTGGCAGCTGAAACACAGTCTCCTTCCCTTTTTGGCTCAACAAAATTACAGCAAGAGTCAAC
GTTTTTGTTTCATGGCAACAAAACTGAAGATACACCTGACAAGAAGATGGAGGTGGCATCTG
AAAAGAAAACGGACCCATCATCACTAGGAGCGACAAGTGCCTCATTTAATTTCGGCAAGAAA
GTTGATAGCTCTGTTTTGGGCTCATTAAGCTCTGTCCCCCTGACTGGATTTTCTTTCTCCCC
TGGAAACTCCAGTTTATTTGGCAAAGATACTACCCAGAGTAAACCAGTCTCTTCACCATTTC
CCACTAAACCATTGGAGGGCCAAGCAGAAGGTGACAGTGGTGAATGCAAAGGTGGAGATGAA
GAAGAGAATGATGAGCCACCCAAAGTAGTAGTTACCGAAGTAAAAGAAGAAGATGCTTTTTA
CTCCAAAAAGTGTAAACTGTTTTACAAGAAAGACAATGAGTTTAAAGAGAAAGGCATAGGTA
CTCTGCATTTAAAACCTACAGCAAATCAGAAGACACAGCTTTTGGTGCGGGCAGACACCAAT
TTAGGCAACATATTGCTGAACGTTCTGATTCCACCCAATATGCCATGTACGCGAACAGGGAA
GAATAACGTTCTTATCGTCTGTGTTCCAAATCCACCAATTGACGAGAAGAATGCCACCATGC
CAGTCACCATGTTGATTCGGGTAAAAACCAGCGAGGATGCAGACGAGTTGCACAAAATTTTA
CTGGAGAAAAAGGATGCCTGA
Nucleic Acid Encoding an Exemplary Nup50 Polypeptide:
(SEQ ID NO: 2)
ATGGCCAAAAGAAATGCCGAGAAGGAACTGACAGATAGGAATTGGGATCAAGAAGATGAAGC
TGAAGAGGTGGGAACATTCTCCATGGCCAGTGAGGAAGTCTTGAAGAATAGAGCCATAAAGA
AAGCAAAGCGCAGAAATGTTGGATTTGAATCTGACACTGGAGGAGCCTTTAAAGGTTTTAAA
GGTTTGGTGGTACCTTCTGGAGGAGGACGCTTTTCTGGATTTGGTAGTGGCGCTGGAGGGAA
GCCTTTGGAAGGACTGTCGAATGGAAACAACATAACCAGTGCCCCTCCCTTCGCCAGTGCAA
AGGCAGCGGCAGATCCCAAGGTAGCCTTTGGTTCTCTTGCTGCAAATGGCCCTACCACCTTG
GTTGATAAAGTTTCAAATCCCAAAACTAATGGGGACAGTCAGCAGCCCTCCTCCTCTGGCCT
TGCTTCCAGTAAAGCTTGTGTCGGAAATGCCTATCACAAGCAGTTGGCCGCCTTGAACTGCT
CCGTGCGGGATTGGATAGTGAAGCACGTGAATACAAACCCCCTCTGTGATCTGACACCTATC
TTTAAAGACTATGAGAAATATTTAGCAAACATTGAACAGCAACACGGGAACAGTGGCAGGAA
TTCTGAAAGTGAATCTAACAAAGTGGCAGCTGAAACACAGTCTCCTTCCCTTTTTGGCTCAA
CAAAATTACAGCAAGAGTCAACGTTTTTGTTTCATGGCAACAAAACTGAAGATACACCTGAC
AAGAAGATGGAGGTGGCATCTGAAAAGAAAACGGACCCATCATCACTAGGAGCGACAAGTGC
CTCATTTAATTTCGGCAAGAAAGTTGATAGCTCTGTTTTGGGCTCATTAAGCTCTGTCCCCC
TGACTGGATTTTCTTTCTCCCCTGGAAACTCCAGTTTATTTGGCAAAGATACTACCCAGAGT
AAACCAGTCTCTTCACCATTTCCCACTAAACCATTGGAGGGCCAAGCAGAAGGTGACAGTGG
TGAATGCAAAGGTGGAGATGAAGAAGAGAATGATGAGCCACCCAAAGTAGTAGTTACCGAAG
TAAAAGAAGAAGATGCTTTTTACTCCAAAAAGTGTAAACTGTTTTACAAGAAAGACAATGAG
TTTAAAGAGAAAGGCATAGGTACTCTGCATTTAAAACCTACAGCAAATCAGAAGACACAGCT
TTTGGTGCGGGCAGACACCAATTTAGGCAACATATTGCTGAACGTTCTGATTCCACCCAATA
TGCCATGTACGCGAACAGGGAAGAATAACGTTCTTATCGTCTGTGTTCCAAATCCACCAATT
GACGAGAAGAATGCCACCATGCCAGTCACCATGTTGATTCGGGTAAAAACCAGCGAGGATGC
AGACGAGTTGCACAAAATTTTACTGGAGAAAAAGGATGCCTGA
Example 2: Exemplary Nup50 Polypeptide Sequences
Exemplary Nup50 Polypeptide (also referred to as Nup50-L):
(SEQ ID NO: 3)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTQLLVRADTNLGNILLNVLIPPNMPCTRTGKNNVLIVCVPNPPI
DEKNATMPVTMLIRVKTSEDADELHKILLEKKDA
Exemplary Nup50 Polypeptide:
(SEQ ID NO: 4)
MASEEVLKNRAIKKAKRRNVGFESDTGGAFKGFKGLVVPSGGGRFSGFGSGAGGKPLEGLSN
GNNITSAPPFASAKAAADPKVAFGSLAANGPTTLVDKVSNPKTINGDSQQPSSSGLASSKACV
GNAYHKQLAALNCSVRDWIVKHVNTNPLCDLTPIFKDYEKYLANIEQQHGNSGRNSESESNK
VAAETQSPSLFGSTKLQQESTFLFHGNKTEDTPDKKMEVASEKKTDPSSLGATSASFNFGKK
VDSSVLGSLSSVPLTGFSFSPGNSSLFGKDTTQSKPVSSPFPTKPLEGQAEGDSGECKGGDE
EENDEPPKVVVTEVKEEDAFYSKKCKLFYKKDNEFKEKGIGTLHLKPTANQKTOLLVRADTN
LGNILLNVLIPPNMPCTRTGKNNVLIVCVPNPPIDEKNATMPVTMLIRVKTSEDADELHKIL
LEKKDA
Exemplary Nup50 Polypeptide (also referred to as Nup50-D16N):
(SEQ ID NO: 14)
MAKRNAEKELTDRNWNQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLFGSTKLQQESTFLFHGNKTEDTPD
KKMEVASEKKTDPSSLGATSASFNFGKKVDSSVLGSLSSVPLIGESFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-E20G):
(SEQ ID NO: 15)
MAKRNAEKELTDRNWDQEDGAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKTNGDSQQPSSSGLASSKACVGNAYHKQLAALNCSVRDWIVKHVNTNPLCDLTPI
FKDYEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-R45C):
(SEQ ID NO: 16)
MAKRNAEKELTDRNWDQEDEAEDVGTFSMASEEVLKNRAIKKAKCRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTQLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-R72C):
(SEQ ID NO: 17)
MAKRNAEKELTDRNWDQEDEAEDVGTFSMASEEVLKNRAIKKAKRRNVGFESDTGGAFKGFK
GLVVPSGGGCFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLFGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTQLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-G114D):
(SEQ ID NO: 18)
MAKRNAEKELTDRNWDQEDEAEDVGTFSMASEEVLKNRAIKKAKRRNVGFESDTGGAFKGFK
GLVVPSGGGRESGFGSGAGGKPLEGLSNGNNITSAPPFASAKAAADPKVAFDSLAANGPTTL
VDKVSNPKINGDSQQPSSSGLASSKACVGNAYHKOLAALNCSVRDWIVKHVNTNPLCDLTPI
FKDYEKYLANIEQQHGNSGRNSESESNKVAAETQSPSLFGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTQLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-Y156C):
(SEQ ID NO: 19)
MAKRNAEKELTDRNWDQEDEAEDVGTESMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNACHKQLAALNCSVRDWIVKHVNTNPLCDLTPI
FKDYEKYLANIEQQHGNSGRNSESESNKVAAETQSPSLFGSTKLQQESTELFHGNKTEDTPD
KKMEVASEKKTDPSSLGATSASENFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-P179A):
(SEQ ID NO: 20)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNALCDLTPI
FKDYEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLFGKDTTOS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-K275E):
(SEQ ID NO: 21)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLFGSTKLQQESTFLFHGNKTEDTPD
KKMEVASEKKTDPSSLGATSASFNFGEKVDSSVLGSLSSVPLTGFSFSPGNSSLFGKDTTOS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-R448W):
(SEQ ID NO: 22)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLEGKDTTOS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIWVKTSEDADELHKILLEKKDA
Exemplary Nup50 Polypeptide (also referred to as Nup50-Y190A):
(SEQ ID NO: 23)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKTNGDSQQPSSSGLASSKACVGNAYHKQLAALNCSVRDWIVKHVNTNPLCDLTPI
FKDAEKYLANIEQQHGNSGRNSESESNKVAAETQSPSLEGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTOLLVRADTNLGNILLNVLIPPNMPCTRTGKNNVLIVCVPNPPI
DEKNATMPVTMLIRVKTSEDADELHKILLEKKDA
Exemplary Nup50 Polypeptide (also referred to as Nup50-E191A):
(SEQ ID NO: 24)
MAKRNAEKELTDRNWDQEDEAEDVGTFSMASEEVLKNRAIKKAKRRNVGFESDTGGAFKGFK
GLVVPSGGGRFSGFGSGAGGKPLEGLSNGNNITSAPPFASAKAAADPKVAFGSLAANGPTTL
VDKVSNPKINGDSQQPSSSGLASSKACVGNAYHKQLAALNCSVRDWIVKHVNTNPLCDLTPI
FKDYAKYLANIEQQHGNSGRNSESESNKVAAETQSPSLFGSTKLQQESTFLFHGNKTEDTPD
KKMEVASEKKTDPSSLGATSASFNFGKKVDSSVLGSLSSVPLTGFSFSPGNSSLFGKDTTQS
KPVSSPFPTKPLEGQAEGDSGECKGGDEEENDEPPKVVVTEVKEEDAFYSKKCKLFYKKDNE
FKEKGIGTLHLKPTANQKTQLLVRADTNLGNILLNVLIPPNMPCTRTGKNNVLIVCVPNPPI
DEKNATMPVTMLIRVKTSEDADELHKILLEKKDA

### Example 3: Nucleoporins can Rescue TDP-43 Proteinopathy in Cellular Models of FTD/ALS

This Example describes the identification of a therapeutic role for one or more nucleoporin polypeptides and/or fragments thereof (and/or nucleic acids designed to express a nucleoporin polypeptide and/or a fragment thereof).

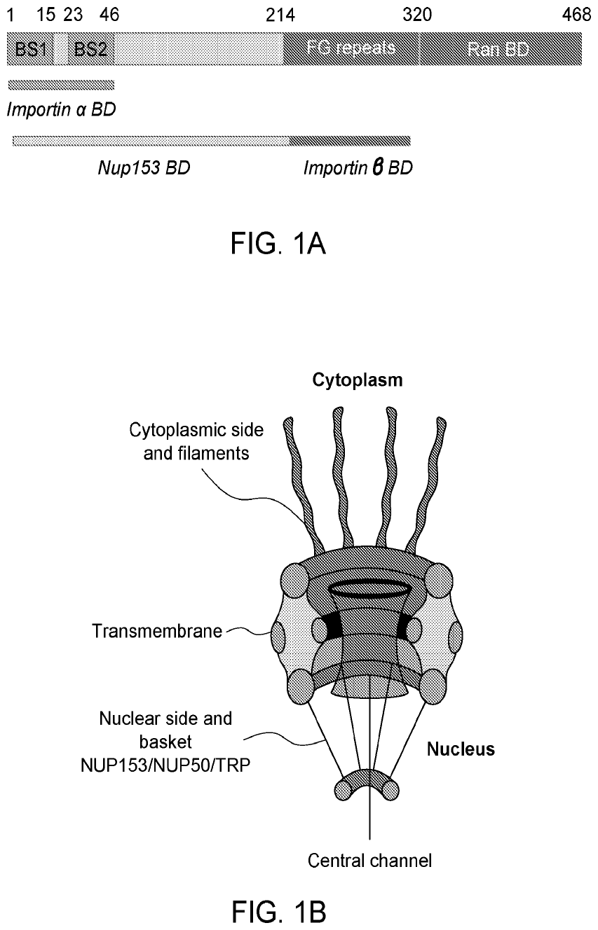

A schematic of the domain structure of a Nup50 polypeptide and its interactions within the nuclear basket and other factors is shown in FIG. 1.

### Specific Nucleoporin Proteins Decrease Insoluble TDP-43 Levels

A comprehensive screen was used to identify nucleoporins having the ability to reduce insoluble TDP-43-mNLS polypeptide levels.

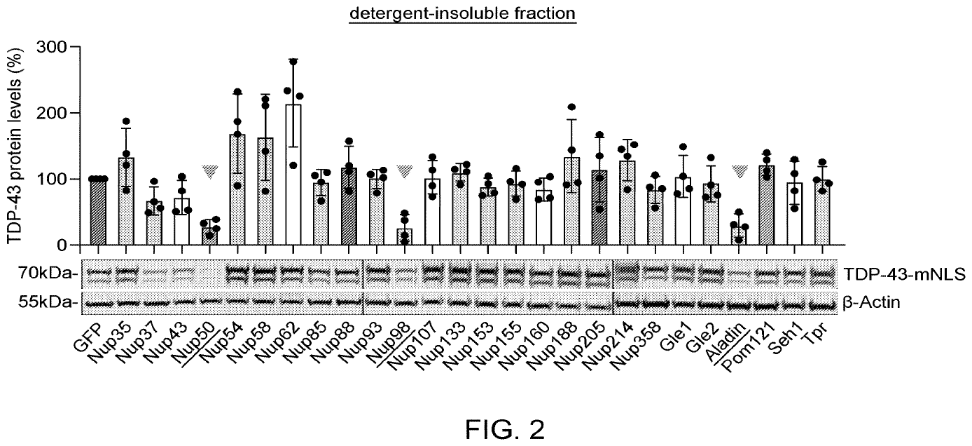

GFP-tagged nucleoporins were coexpressed with mCherry-tagged TDP-mNLS in HEK293 cells, and RIPA-buffer-insoluble fractions were immunoblotted and stained for TDP-43 and were also stained for β-actin as a loading control (FIG. 2, bottom). The TDP-43 signal was normalized to the loading control signal, and then compared to the GFP control signal (FIG. 2, top).

A significant reduction of insoluble TDP-43-mNLS was observed for Nup50 polypeptides, Nup98 polypeptides, and Aladin polypeptides (arrowheads in FIG. 2, top). n=3, One-way ANOVA was used to test for significance, p<0.05.

### Nup50 and Nup98 Expression Decrease Insoluble TDP-43 Levels

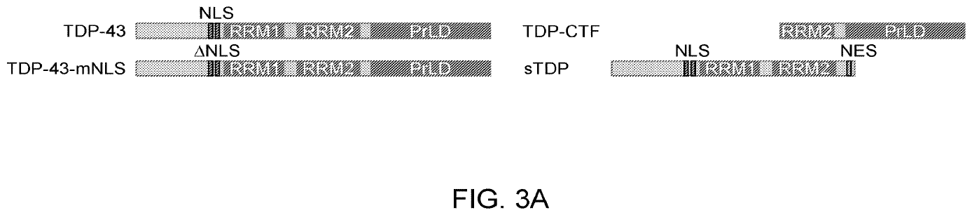

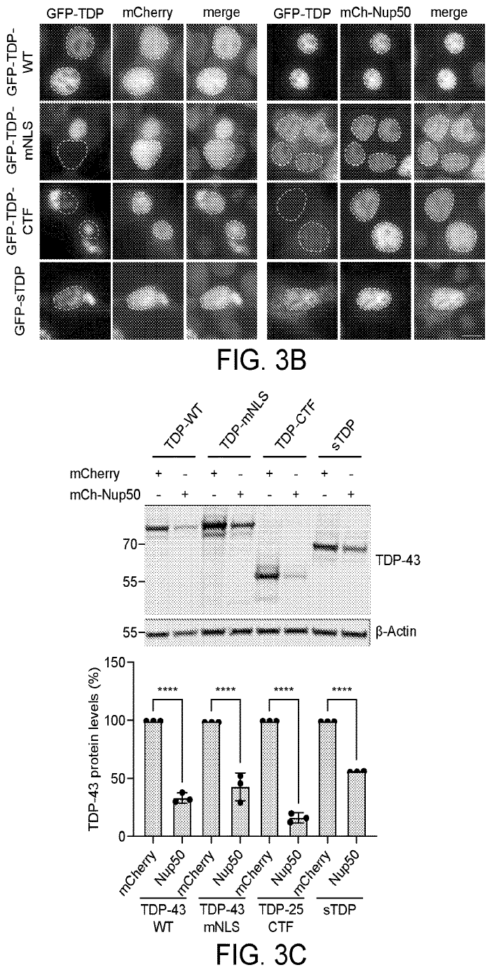

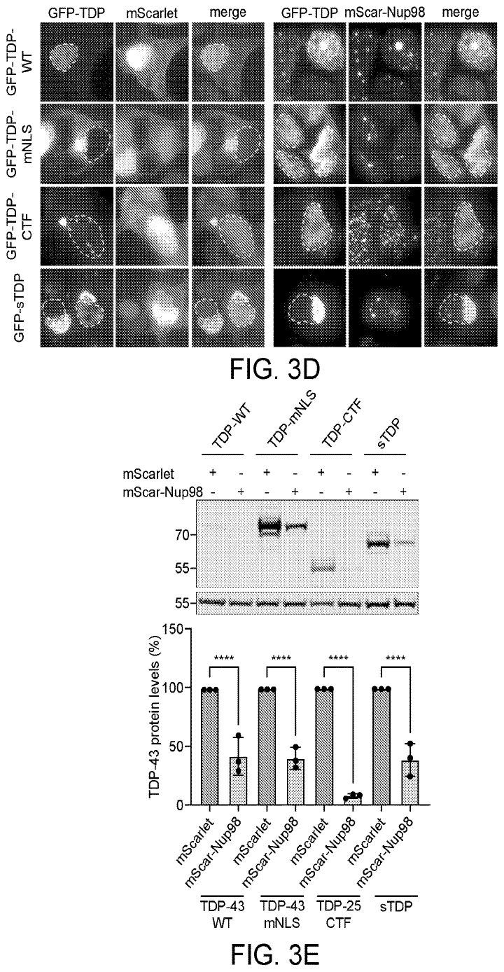

Schematics of TDP-43 constructs used as shown in FIG. 3A. mCherry-tagged Nup50 (FIG. 3B) and mScarlet-tagged Nup98 (FIG. 3D) were coexpressed with GFP-tagged TDP-43 constructs in HEK293 cells. Hoechst staining was used to trace nuclei (FIG. 3B and FIG. 3D). RIPA-buffer-insoluble fractions of HEK293 cells co-expressing GFP-tagged TDP-43 constructs with either mCherry-tagged Nup50 (FIG. 3C top) or mScarlet-tagged Nup98 (FIG. 3E top) were immunoblotted for TDP-43 polypeptides. Immunoblotting for β-actin was used as a loading control. Quantification of TDP-43 signal normalized to loading control and compared to fluorescent control treatment (FIG. 3C bottom and FIG. 3E bottom). n=3, One-way ANOVA was used to test for significance, ****p<0.0001.

These results demonstrate that Nup50 polypeptides and Nup98 polypeptides can reduce cytoplasmic TDP-43 aggregation.

Nup50 and Nup98 eliminate pathological phospho-TDP-43S^{409/410}

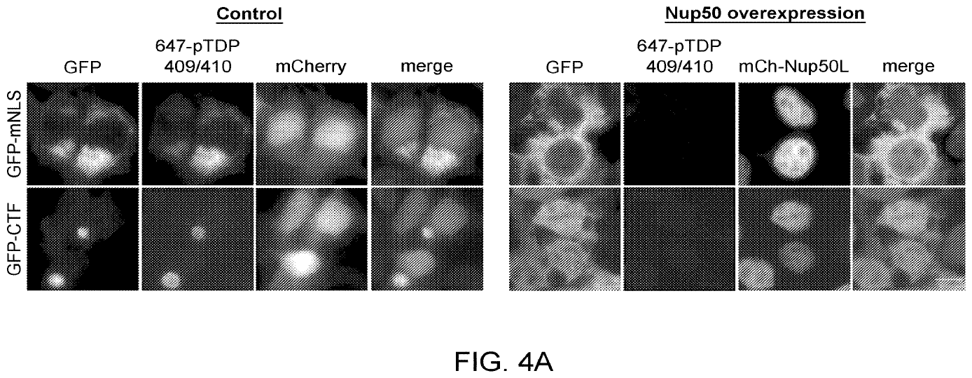

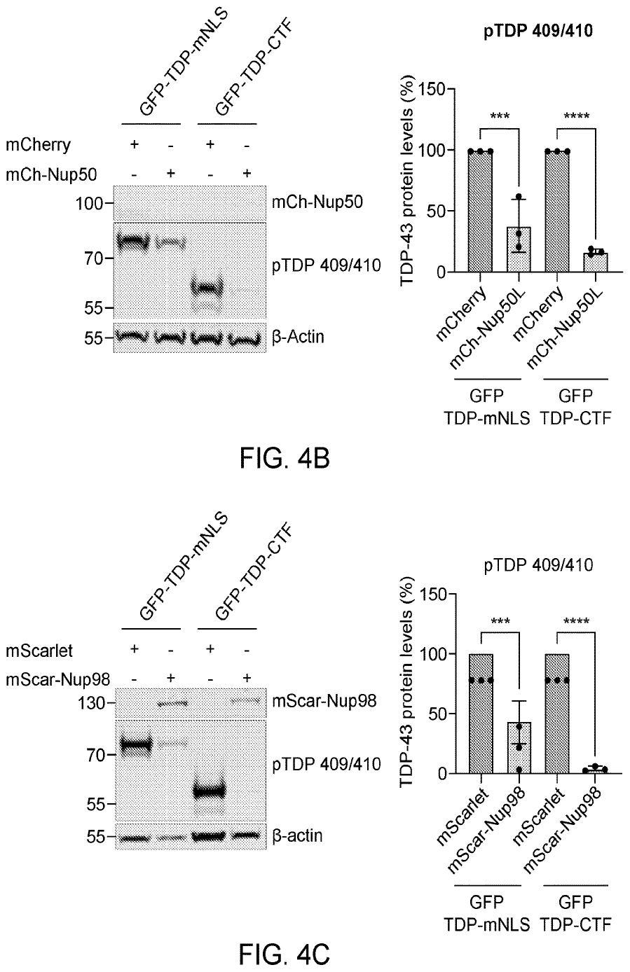

mCherry-tagged Nup50 was coexpressed with GFP-tagged TDP-43 constructs, and immunocytochemistry staining for TDP-43 phosphorylated at S409/410 was performed (FIG. 4A). RIPA-buffer-insoluble fractions of HEK293 cells coexpressing GFP-tagged TDP-43 constructs with either mCherry-tagged Nup50 or mScarlet-tagged Nup98 were immunoblotted for Nup50 polypeptides (FIG. 4B, left) or Nup98 polypeptides (FIG. 4C. left), TDP-43 phosphorylated at S409/410, and β-actin as a loading control. The TDP-43 polypeptide signal was normalized to loading control and compared to mCherry (FIG. 4B, right) or mScarlet (FIG. 4C, right) as a negative control. n=3, One-way ANOVA to test for significance ***p=0.0001, ****p<0.0001.

These results demonstrate that that Nup50 polypeptides and Nup98 polypeptides can reduce the accumulation of pathological hyperphosphorylated TDP-43.

### Nup50 Activity Towards TDP-43 Resides in a Short α-Helical Domain

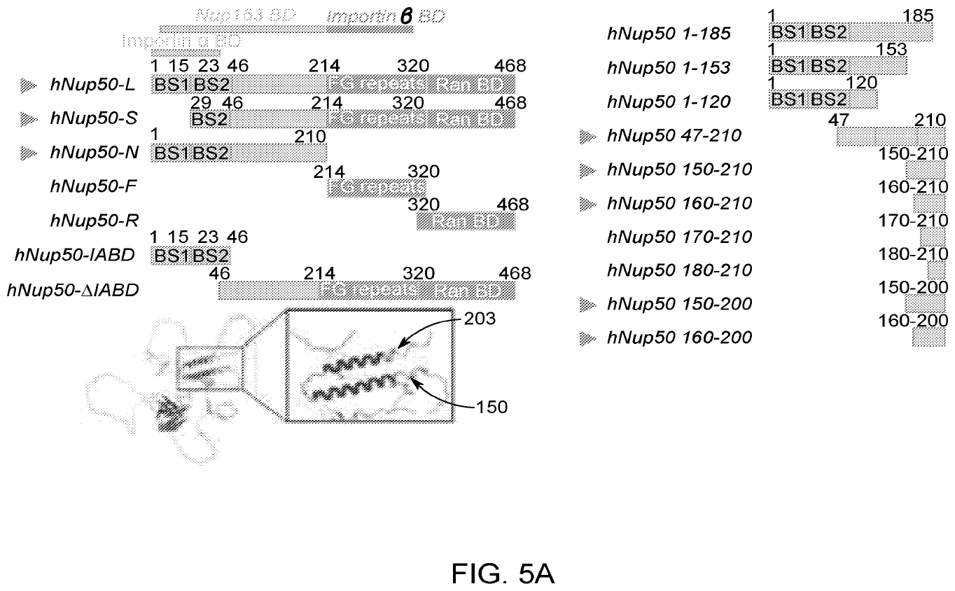

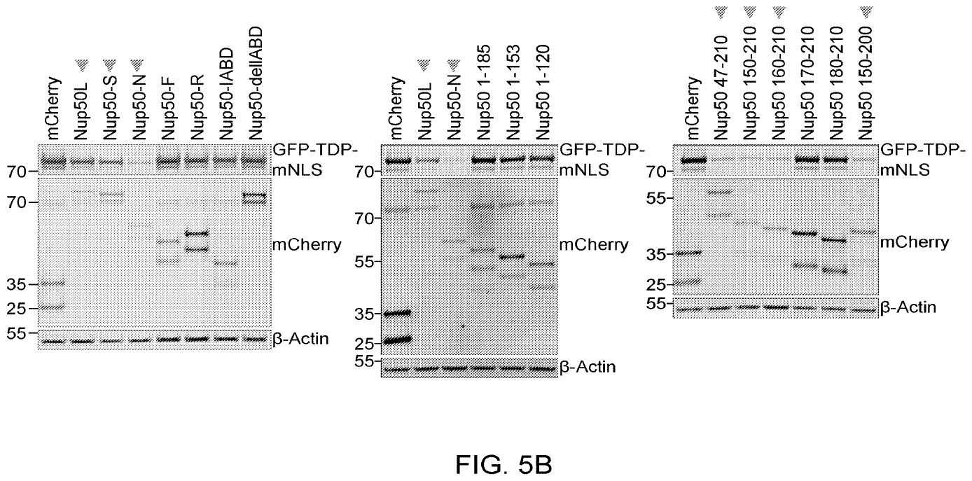

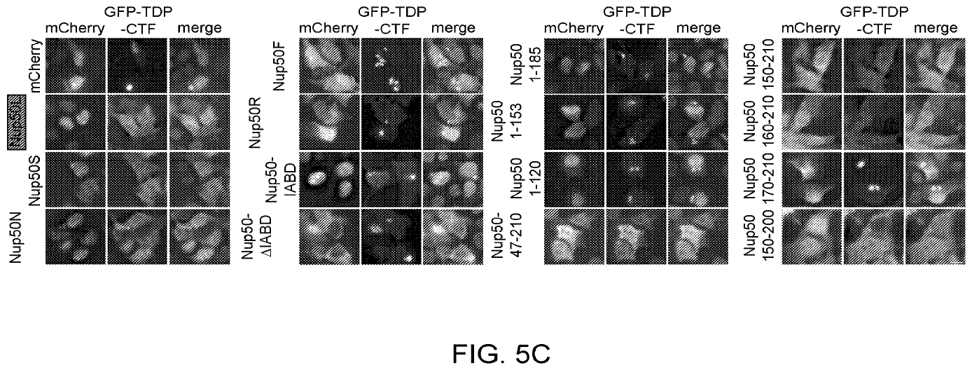

A schematic of Nup50 polypeptide fragments and truncations is shown in FIG. 5A. An AlphaFold structure of Nup50 highlighting double alpha-helices within the smallest active fragment is also shown in FIG. 5A. RIPA-insoluble fractions from HEK293 cells coexpressing various mCherry-tagged Nup50 polypeptide fragments and GFP-tagged TDP-mNLS were immunoblotted for TDP-43 and mCherry (FIG. 5B). β-actin was used as a loading control. Arrowheads indicate constructs that decreased insoluble TDP-mNLS. Representative examples for the coexpression of mCherry-tagged Nup50 constructs and GFP-tagged TDP-CTF are shown in FIG. 5C.

These results demonstrate that the N-terminus of a Nup50 polypeptide was effective and sufficient for reducing cytoplasmic TDP-43 aggregates.

### Example 4: Nup50 as a Risk Factor and Therapeutic Target for ALS

The results in this Example re-present and expand on at least some of the results provided in other Examples.

### Nup50 Expression Rescues TDP-43 Pathology

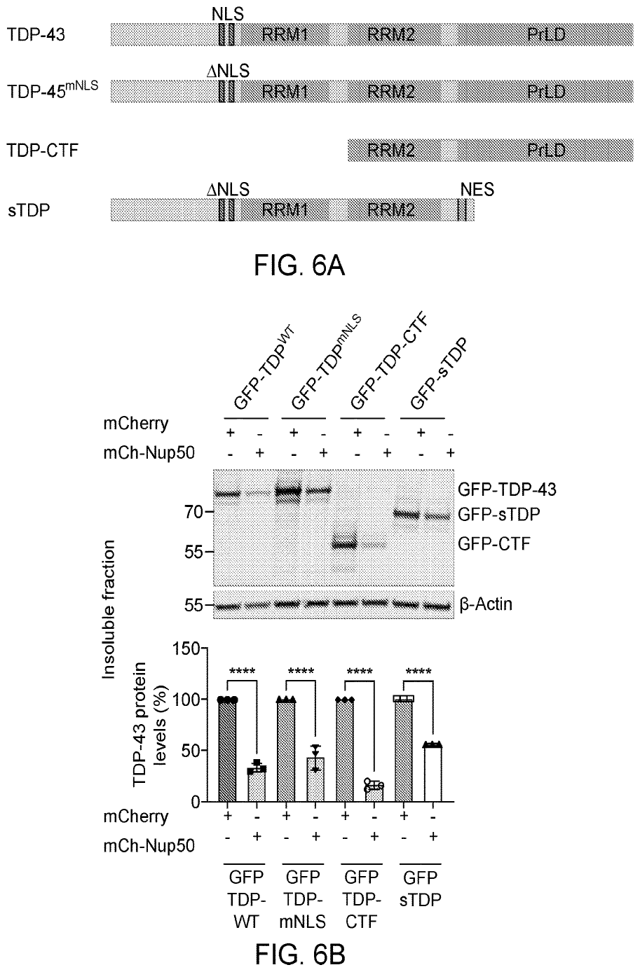

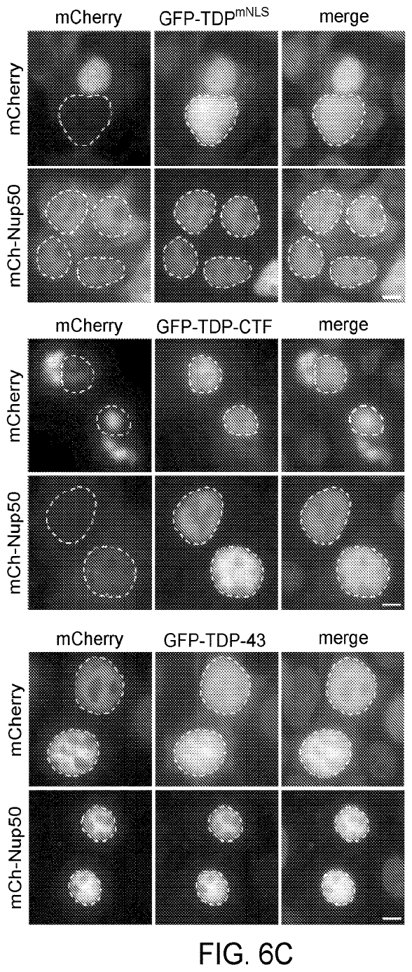

A comprehensive screen of twenty-six components of nuclear pore complexes (nucleoporins) was performed to identify additional factors affecting TDP-43 pathology. Schematics of the GFP-tagged TDP-43mNLS, TDP-CTF, and sTDP construct used are shown in FIG. 6A. Quantitative western blot analyses showed that Nup50 polypeptides significantly reduced the RIPA-buffer insoluble levels of TDPCTF, TDP-43mNLS, and to a lesser degree sTDP (FIG. 6B). mCherry-NUP50 suppressed pathological GFP-TDP-CTF and GFP-TDP-43mNLS aggregates and restored their nuclear localization, despite the lack of a functional NLS, while wild type GFP-TDP-43 remains nuclear and soluble (FIG. 6C). Endogenous TDP-43 polypeptides were not affected. Expression of Nup50 polypeptides reduced TDP-43 polypeptide aggregation.

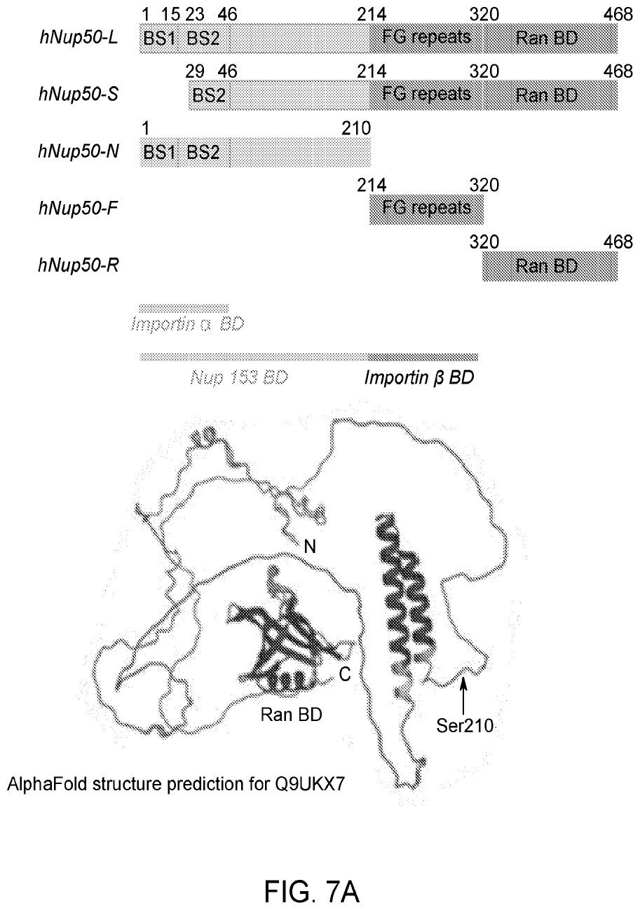

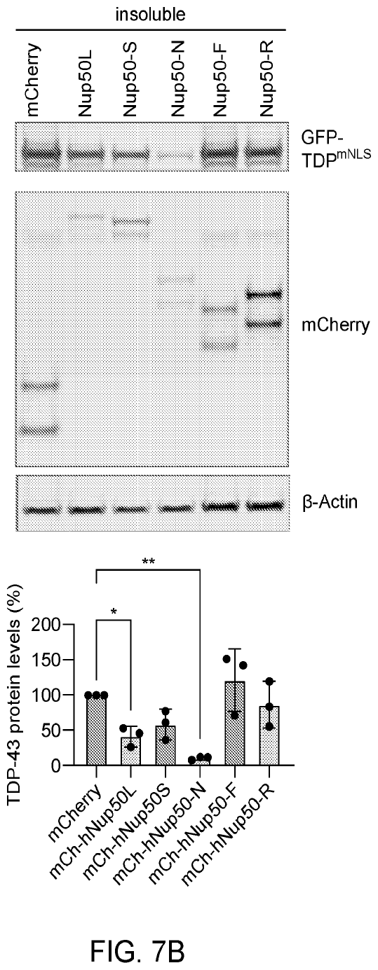

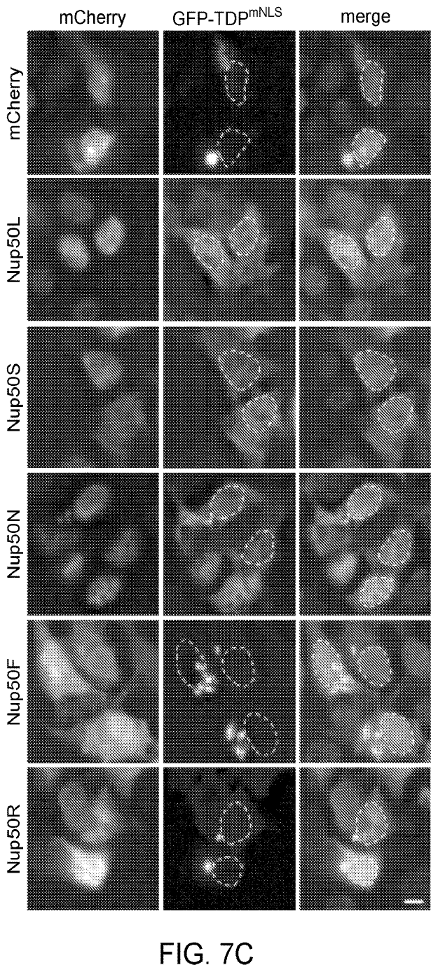

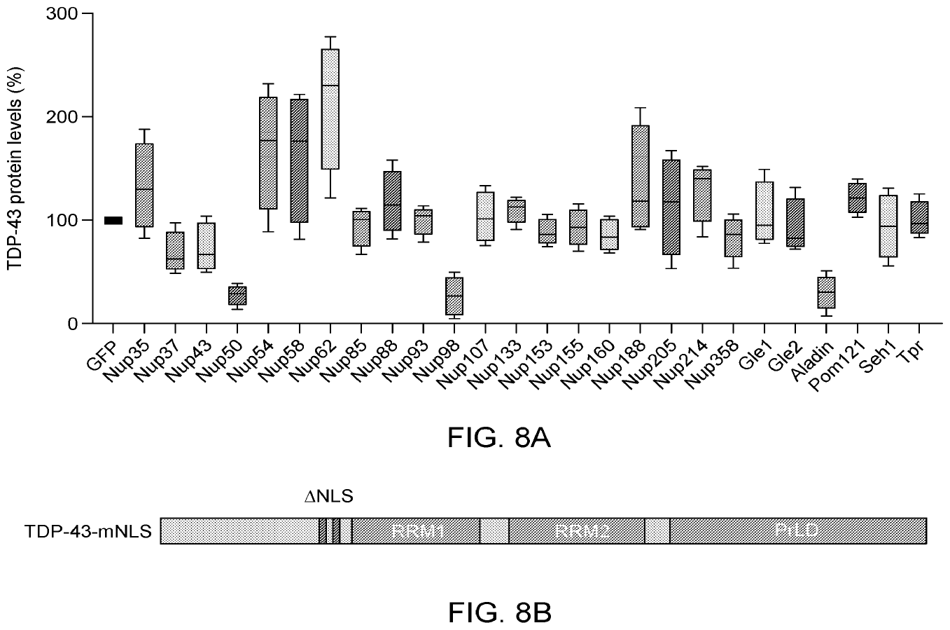

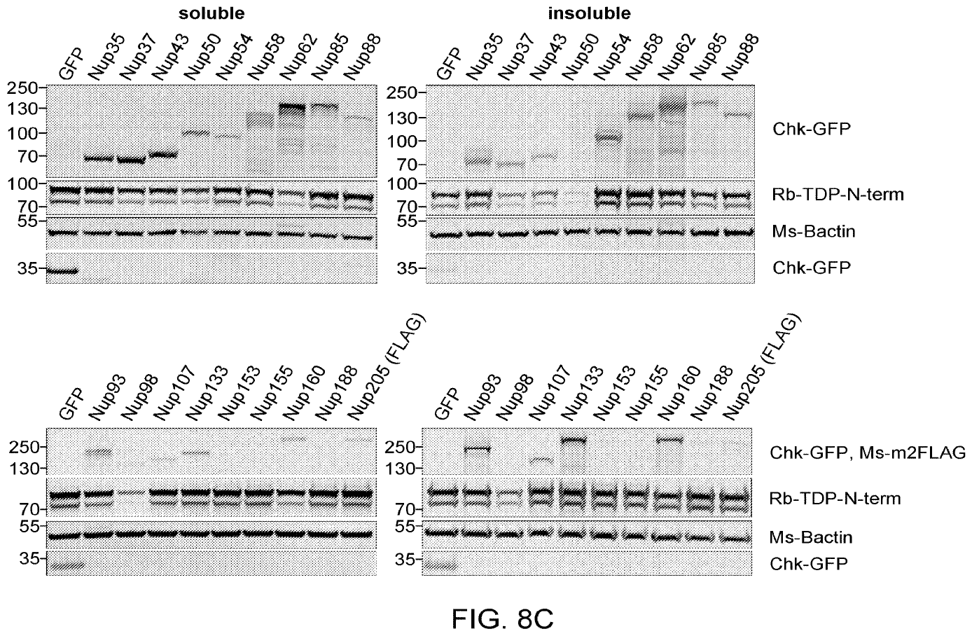

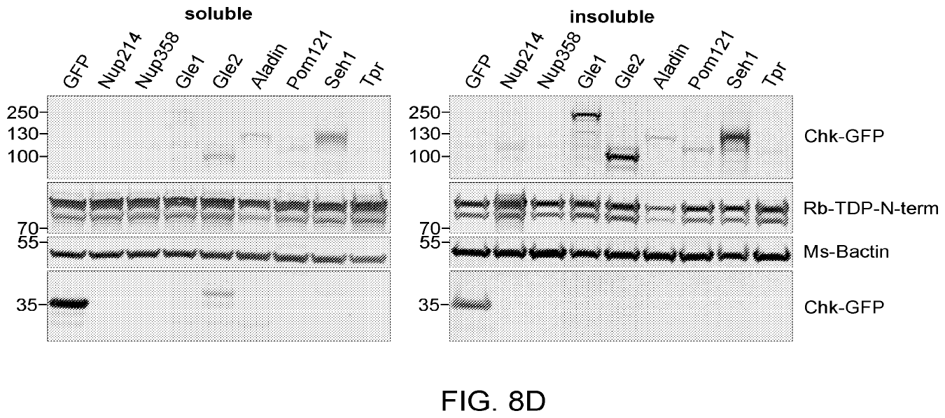

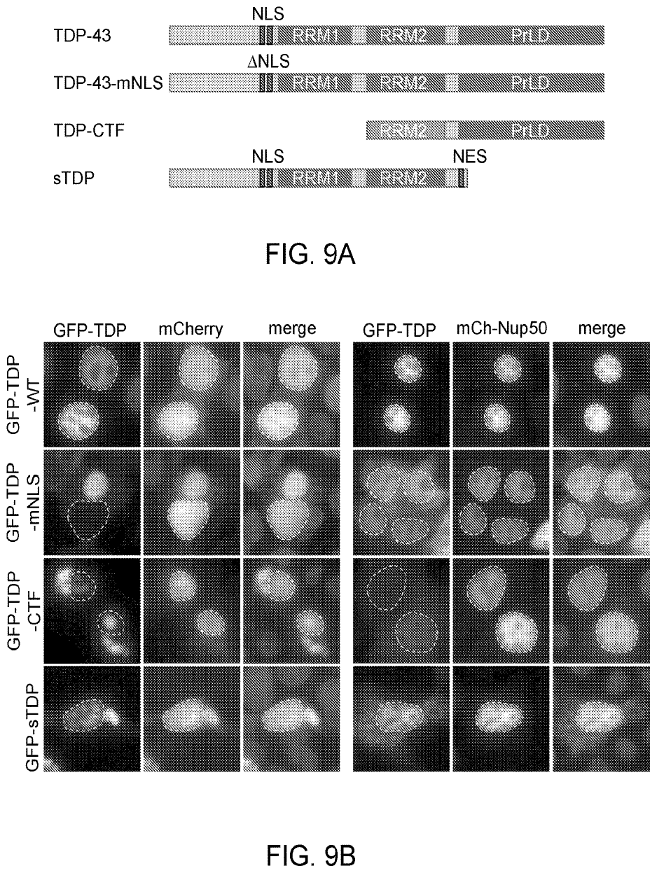

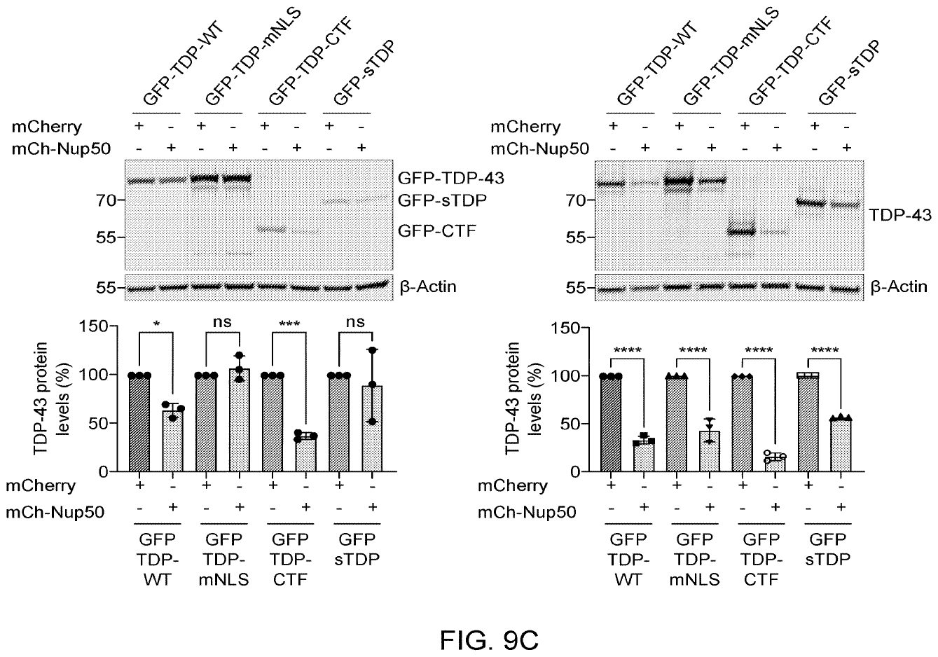

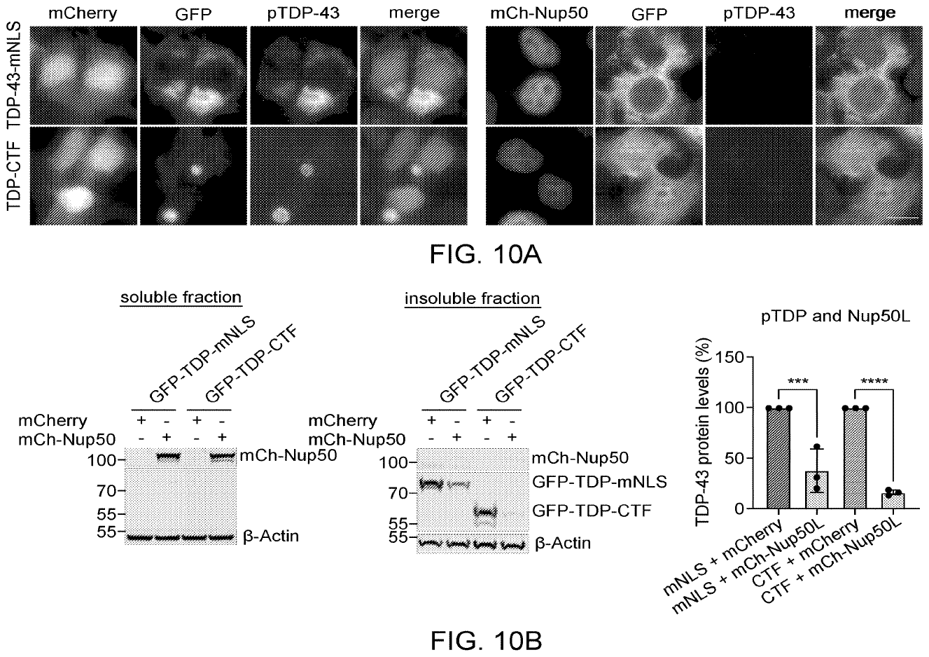

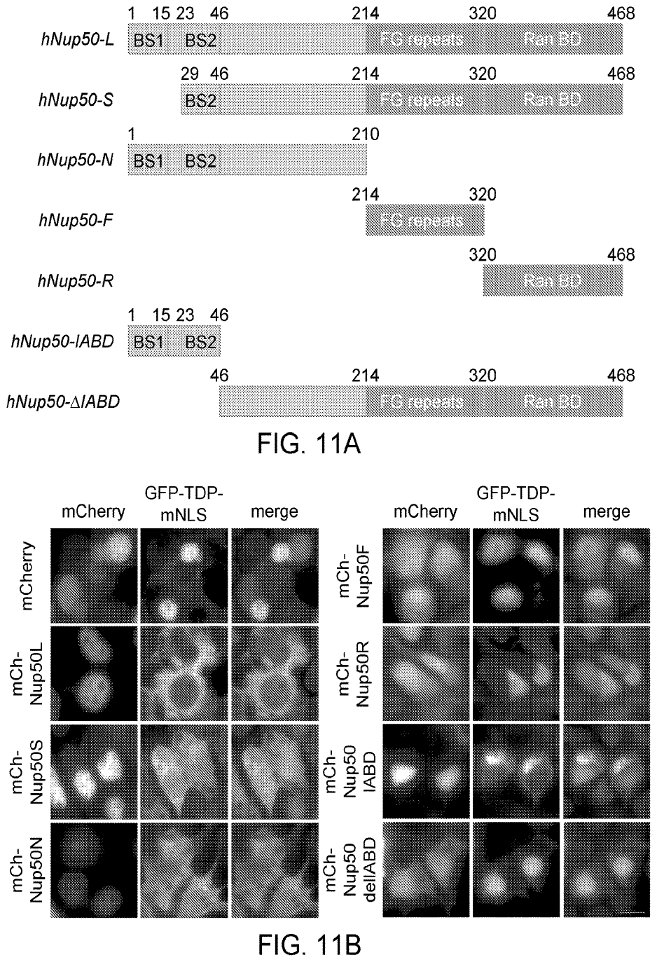

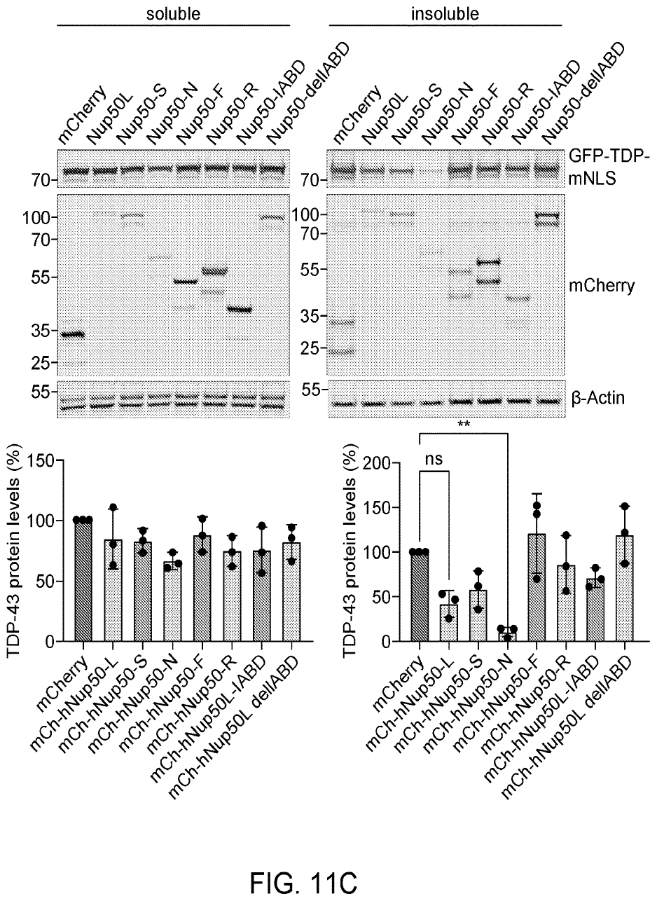

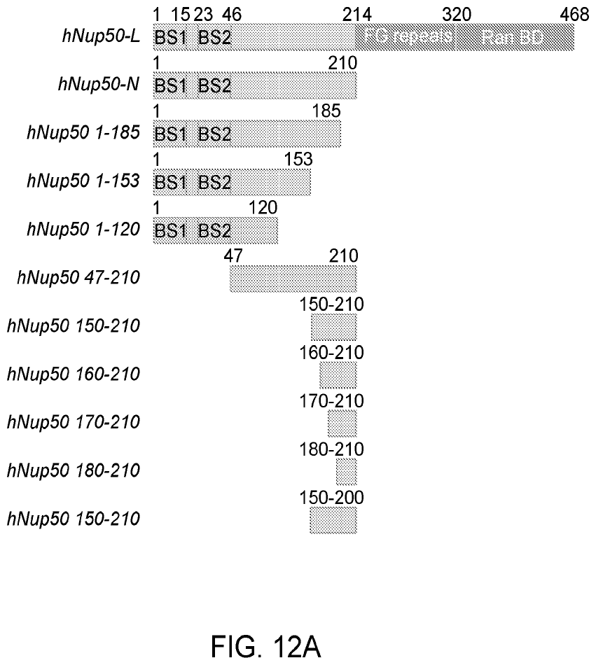

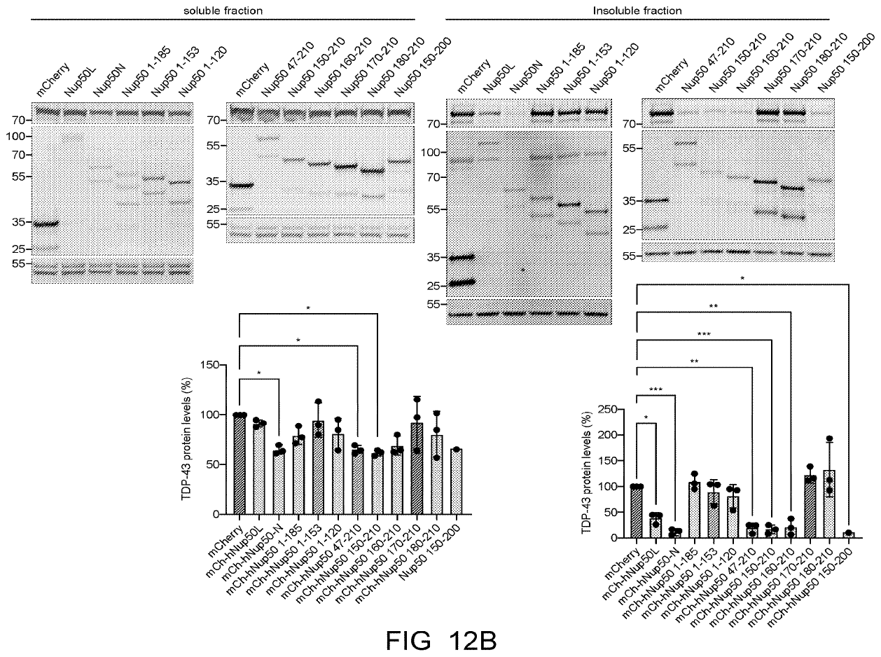

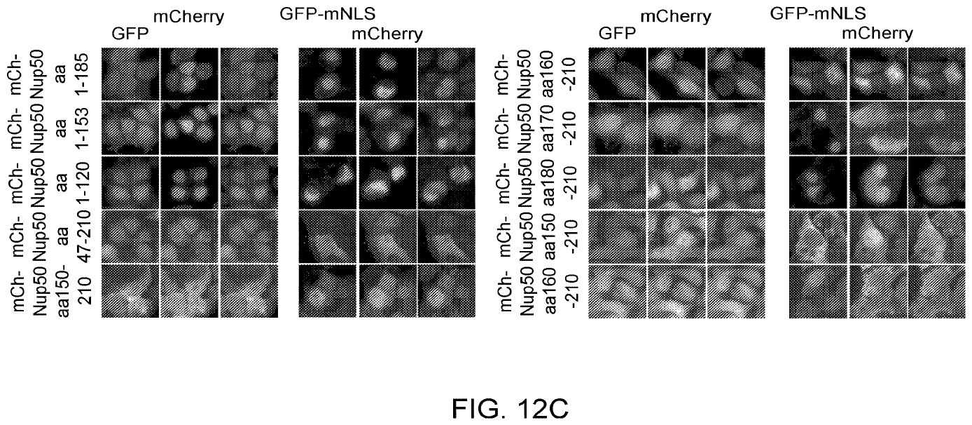

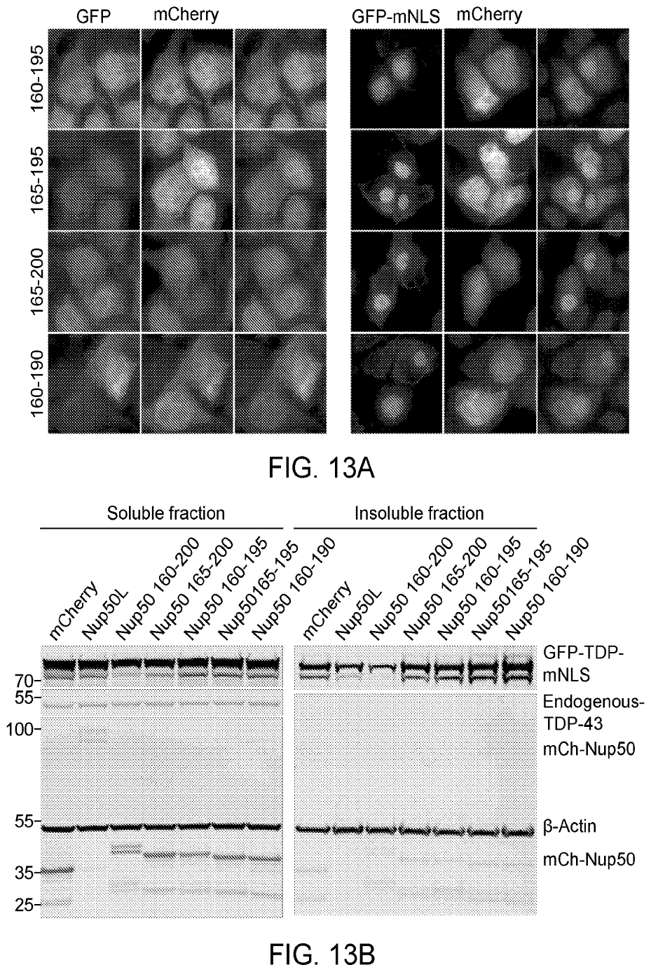

To identify the region of a Nup50 polypeptide sufficient to rescue TDP-43 pathology, an extensive series of Nup50 polypeptide truncations was generated (FIG. 7A). The Nup50 variants either abolished or enhanced the chaperone activity (FIGS. 7B and 7C). It was found that the N-terminal fragment (aa 1-210) of a Nup50 polypeptide was more active than a full-length Nup50 polypeptide in reducing detergent-insoluble TDP-43^{mNLS} levels.

These results demonstrate that Nup50 polypeptides and fragments thereof can be used to rescue TDP-43 pathology in ALS.

### Example 5: Nup50 Polypeptides Decrease Insoluble TDP-43-mNLS

The results in this Example re-present and expand on at least some of the results provided in other Examples.

### Methods

### Mammalian Cell Culture and Transfection

HEK293T cells were purchased from ATCC (CRL-1573). Human HEK293T cells were cultured in DMEM media (Life Technologies) containing 10% FBS and transfected with Lipofectamine LTX (Invitrogen cat #15338100) according to the manufacturer's protocol.

### Immunocytochemistry and Image Acquisition

Cells were seeded 24 hours prior to transfection in 24-well plates with poly-lysine-coated coverslips. Fresh media was exchanged 24 hours post transfection. Cells are fixed 48 hours post-transfection with warm 4% paraformaldehyde (PFA) for 15 minutes. For immunostaining experiments, cells were permeabilized for 10 min with Triton X-100 in PBS, blocked for 45 minutes in 5% bovine serum albumin (BSA) in PBS. Cells were incubated with primary antibodies diluted in 5% BSA in PBS for 1 hour at room temperature (RT) or overnight at 4° C. Cells were washed with PBS-T before incubation with fluorophore-coupled secondary antibodies for 45 minutes at room temperature. Cells were counterstained with Hoechst for 10 minutes at room temperature and mounted onto slides using Prolong Glass (Invitrogen cat #P36980).

High-resolution fluorescence images were acquired using a Nikon Eclipse Ti2 epifluorescence microscope equipped with an Andor Zyla sCMOS camera. Within each experiment single and multiple z-plane images were acquired with the same acquisition settings. Image stacks were deconvolved using a 3D blind constrained iterative algorithm (Nikon NIS Elements) over 10 iterations.

### Cell Lysis and Subcellular Fractionation

Cells were seeded 24 hours prior to transfection in 12-well plates. Cells were transfected and media was exchanged after 24 hours. 48 hours post-transfection cells were lysed using RIPA Lysis and Extraction buffer (Thermo Fisher Scientific) supplemented with a protease inhibitor cocktail (Roche) and centrifuged at 15000 rpm for 20 minutes at 4° C. The supernatant was collected as the detergent-soluble fraction. Urea buffer (7M urea, 2M thiourea, 4% CHAPS, 50 mM Tris pH 8, Roche complete protease inhibitor cocktail) was added to the insoluble pellet. The samples were briefly sonicated and left at room temperature for 30 minutes and centrifuged at 15000 rpm for 20 minutes at 4° C. The supernatant was then collected as the detergent-insoluble fraction.

### Western Blot Analysis

For western blot analysis, the samples were boiled in 1× Laemmli sample buffer for 5 min at 98° C. and run on Bolt™ 4 to 12% Bis-Tris gels (Thermo Fisher Scientific) at 100 mV. Proteins were transferred to nitrocellulose membranes using an iBlot™ 2 Gel Transfer device (Thermo Fisher Scientific). Membranes were blocked with Odyssey blocking buffer (LI-COR) for 1 hour at room temperature followed by incubation with the following primary antibodies overnight at 4° C. Membranes were washed in PBS-T and incubated with secondary antibodies (at 1:10,000) in PBS blocking buffer with 0.05% Tween 20 for 45 minutes at room temperature. Blots were imaged using an Odyssey scanner (LI-COR). Images were quantified using LI-COR Empiria Studio software.

### Statistics

Statistical comparisons between experimental groups were performed using either two-sided Student's t-test or analysis of variance (one- or two-way ANOVA followed by Bonferroni's post hoc test) in GraphPad Prism software (version 9.2.0). Differences were considered statistically significant when p<0.05. Data is shown as mean±SEM.

### Example 6

The results in this Example re-present and expand on at least some of the results provided in other Examples.

### Methods

### Mammalian Cell Culture and Transfection

HEK293T cells were purchased from ATCC (CRL-1573). Human HEK293T cells were cultured in DMEM media (Life Technologies) containing 10% FBS and transfected with Lipofectamine LTX (Invitrogen cat #15338100) according to the manufacturer's protocol.

### Immunocytochemistry and Image Acquisition

Cells were seeded 24 hours prior to transfection in 24-well plates with poly-lysine-coated coverslips. Fresh media was exchanged 24 hours post transfection. Cells were fixed 48 hours post-transfection with 4% paraformaldehyde (PFA) for 15 minutes at room temperature. Cells were counterstained with Hoechst for 10 minutes at RT and mounted onto slides using Prolong Glass (Invitrogen cat #P36980). For NPC imaging, cells were fixed 24 hours after transfection with a rapid 0.2% Triton X-100 in PBS pretreatment before fixation with PFA to reduce diffuse nuclear fluorescence signal.

High-resolution fluorescence images were acquired using a Nikon Eclipse Ti2 epifluorescence microscope equipped with an Andor Zyla sCMOS camera. Within each experiment single and multiple z-plane images were acquired with the same acquisition settings. Image stacks were deconvolved using a 3D blind constrained iterative algorithm (Nikon NIS Elements, version 5.30.03) over 10 iterations.

### Cell Lysis and Subcellular Fractionation

Cells were seeded 24 hours prior to transfection in 12-well plates. Cells were transfected and media was exchanged after 24 hours. Forty-eight hours post-transfection, cells were lysed using RIPA Lysis and Extraction buffer (Thermo Fisher Scientific) supplemented with a protease inhibitor cocktail (Roche) and centrifuged at 15000 rpm for 20 minutes at 4° C. The supernatant was collected as the detergent-soluble fraction. Urea buffer (7 M urea, 2 M thiourea, 4% CHAPS, 50 mM Tris pH 8, Roche complete protease inhibitor cocktail) was added to the insoluble pellet. The samples were briefly sonicated and left at RT for 30 minutes and centrifuged at 15000 rpm for 20 minutes at 4° C. The supernatant was then collected as the detergent-insoluble fraction. For experiments with the total protein lysate, cells were lysed and collected directly in Urea buffer, briefly sonicated, and centrifuged at 15000 rpm for 20 minutes. The supernatant was then collected as the total protein fraction.

### Immunoprecipitation (IP) and On-Bead Digestion for MS/MS

Cells were seeded in 6-well plates, with 2 wells for each condition. Following 48 hours of transfection, cells were washed twice with PBS and collected in Lysis Buffer (10 mM Tris/Cl pH7.4 (j60202.K2), 150 mM NaCl, 0.5 mM EDTA (Fisher J15694.AE), 0.05% NP-40 (Fisher #28324). Each sample was sonicated for 2 seconds and incubated on ice for 30 minutes with periodic agitation. Samples were centrifuged at 16,500×g for 10 minutes at 4° C. Supernatants were collected, and a portion was saved as the input for quality control. Magnetic agarose GFP-TRAP beads (Chromotek) were prepared by washing with ice cold dilution buffer (10 mM Tris/Cl, 150 mM NaCl, 0.05% NP-40, 0.5 mM EDTA) and collected with a magnet. Samples were diluted with dilution buffer (10 mM Tris/Cl pH7.4, 150 mM NaCl, 0.5 mM EDTA) and added to GFP-TRAP beads. Samples were incubated with beads and rotated overnight at 4° C. A sample of unbound lysate was collected for quality control. Beads were washed with wash buffer (10 mM Tris/Cl pH7.5, 150 mM NaCl, 0.05% NP-40, 0.5 mM EDTA) at room temperature. 15% of beads were saved for western blot analysis of IP.

On-bead digestion was performed according to Chromotek guidelines. Beads were resuspended in lysis buffer and centrifuged at 2,500×g at 4° C. for 2 minutes. Beads were resuspended in elution buffer I (Tris/HCl pH 7.4, 5 g/mL Sequencing Grade Modified Trypsin (Promega), 1 mM DTT) and incubated at 30° C. at 400 rpm for 30 minutes. Samples were centrifuged for 2 minutes, and the supernatant was collected. Beads were resuspended in elution buffer II (50 mM Tris/HCl pH 7.5, 2M Urea, 5 mM iodoacetamide), centrifuged, and the supernatant was combined with the supernatant from the first elution. Digests were incubated overnight at 32° C. at 400 rpm and the reaction was stopped with addition of trifluoroacetic acid. Dried samples were sent for MS/MS. For WB analysis of GFP-TRAP IP samples, beads were incubated in 1× Laemmli sample buffer for 5 minutes at 95° C. and the supernatant was loaded onto an SDS-page gel. For proteomics analysis, proteins were subjected to tryptic digest and peptides were separated using Evosep One LC system and analyzed in Bruker TimsTof Pro 2 mass spectrometer in diaPASEF acquisition mode. Peptide search and summarization of protein intensities per sample was done using the Spectronaut 18 software (Biognosys). Analysis of proteomic data including pre-filtering of protein set, variance normalization, imputation of missing values and statistical comparisons of protein intensities between the studied conditions was done using DEP R package. Plots and heatmaps were generated using DEP, ggplot2 and pheatmap R packages.

### Western Blot Analysis

Protein samples were boiled in 1× Laemmli sample buffer for 5 minutes at 98° C. and run on Bolt™ 4-12% Bis-Tris gels (Thermo Fisher Scientific). Proteins were transferred to nitrocellulose membranes using an iBlot™ ⅔ Gel Transfer device (Thermo Fisher Scientific). Membranes were blocked with Odyssey blocking buffer (LI-COR) for 1 hour at RT followed by incubation with the following primary antibodies: anti-B-actin (1:1250), anti-mCherry (1:5000), anti-GFP (1:5000), anti-Nup50 (1:1250), anti-TDP-43 (1:1250), or anti-pTDP-43^{s409/410} (1:5000) overnight at 4° C. Membranes were washed in PBS-T and incubated with secondary antibodies (1:5000) in PBS blocking buffer with 0.05% Tween 20 for 1 hour at RT. Blots were imaged using an Odyssey scanner (LI-COR) and (version 5.2, LI-COR). Images were quantified using LI-COR Empiria Studio software (version 5.2, LI-COR).

### Ex Vivo Mouse Organotypic Brain Slice Preparation

Brain slices cultures (BSCs) were generated from postnatal day 8-9 (P8-9) C57BL/6 mice. Pups were placed in an isofluorane chamber and decapitated after confirmation of a negative toe-pinch response. Hemi-brains were dissected, and the cortex and hippocampus were collected in sterile, filtered ice-cold dissection buffer: Hank's balanced salt solution (HBSS), calcium, magnesium, no phenol red (Thermo Fisher Scientific), 2 mM ascorbic acid (Sigma Aldrich), 39.4 μM ATP (Sigma Aldrich) and 1% (v/v) penicillin/streptomycin (Thermo Fisher Scientific). Hemi-brains were placed on filter paper and cut using a McIllwain™ tissue chopper (Stoelting Co.) into 350 m slices. Slices were placed, 3 slices per well, in 6-well tissue culture plates with semi-porous membrane insets (0.4 μm pore diameter, Thermo Fisher Scientific, PICM03050). BSCs were incubated at 37° C. and 5% CO₂ in sterile-filtered culture medium containing Basal Medium Eagle (Thermo Fisher Scientific), 26.6 mM HEPES (pH 7.1, Thermo Fisher Scientific), 511 μM ascorbic acid, 1% (v/v) GlutaMAX (Thermo Fisher Scientific), 0.033% (v/v) insulin (Sigma Aldrich), 1% (v/v) penicillin/streptomycin (Thermo Fisher Scientific) and 25% (v/v) heat-inactivated horse serum (Sigma Aldrich). Fresh culture medium was changed every 3 days.

### Production of Minimally-Purified Adeno-Associated Virus (AA V)

rAAV 8 expressing GFP, GFP-TDPmNLS, or GFP-TDP-CTF, under the control of the hCAG promoter, were generated. Hek293T cells were seeded in 6-well plates overnight and then transfected with the transgene plasmid packaged with rAAV8 (pDP8.ape; Plasmid Factory GmbH & Co.KG) using Polyethylenimine Linear (Polysciences). Microscale virus was collected by centrifugation of the supernatant media at 500 g for 5 minutes 72 hours after transfection. rAAVs were applied to BSCs by addition to the culture media on the first day of tissue culture collection (0 DIV).

### Immunofluorescence on Organotypic BSC

BSCs were briefly washed with PBS then fixed with 4% PFA for 1 hour. Individual slice cultures were then cut out from their membranes and treated as free-floating sections. BSCs were permeabilized for 18 hours in 0.2% PBS-Tx on a shaker at 4° C., placed in 20% BSA (Sigma Aldrich) for 3 hours at RT. Primary antibodies in 5% BSA/PBS were applied on slice cultures for 48 hours at 4° C. on a rocker. BSCs were washed and incubated with fluorophore-coupled secondary antibodies for 3 hours are RT. Slices were washed twice with PBS, incubation in Hoechst (1:5000 in PBS) for 25 minutes, and then washed a final time before mounting on slides with Prolong Gold Antifade (Thermo Fisher).

### Results

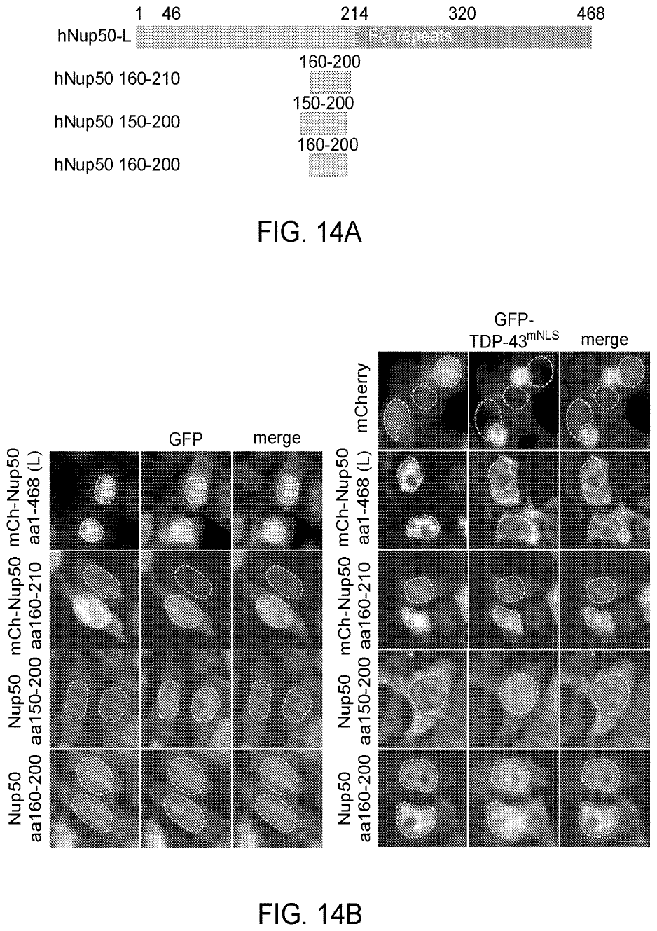

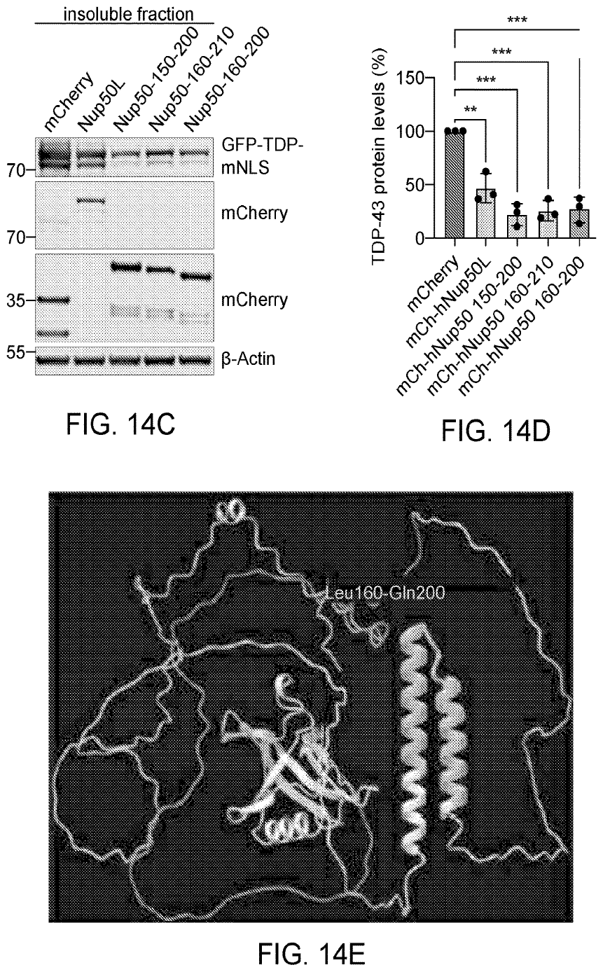

The ability of Nup50 to reduce pathological TDP-43 aggregation was mapped to a 41 amino acid (aa) fragment. This 41aa fragment, within the Nup153-binding domain of Nup50, is sufficient to reduce pathological TDP-43 aggregation via ICC and WB (FIGS. 14A-14D). The smallest active fragment corresponds to a double alpha-helical secondary structure in Nup50 (FIG. 14E).

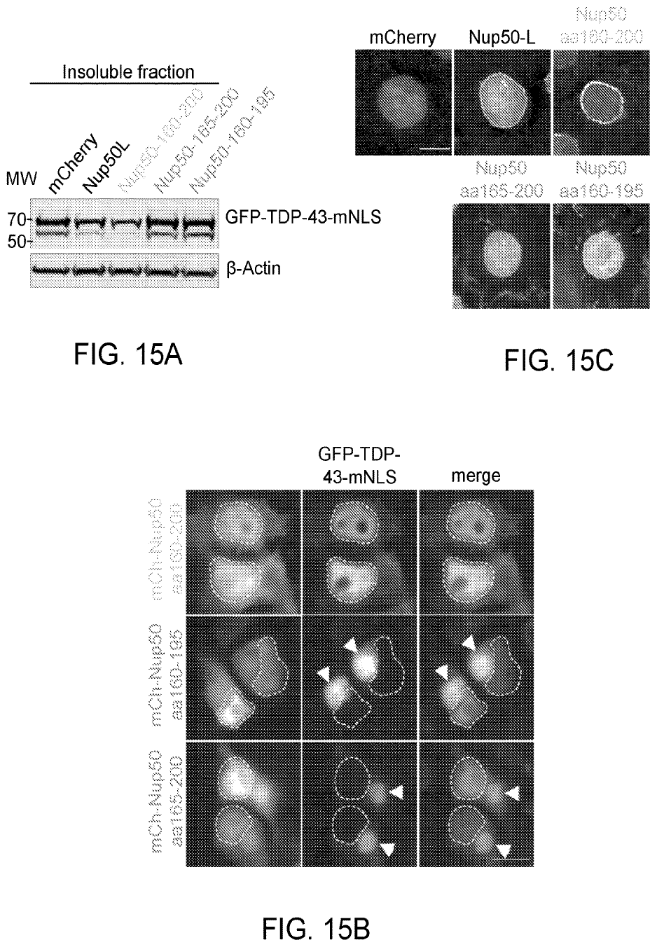

A Xenopus Nup50 polypeptide fragment similar to an active human NUP50 polypeptide fragment has been shown to be required for NPC binding (Holzer et al., The EMBO Journal 40:e108788 (2021)). Further truncation of aa160-200 by 5 amino acids (aa160-195 or aa165-200) abrogates its ability to reduce pathological TDP-43 aggregation (FIGS. 15A and 15B). The smallest active fragment of Nup50 (aa160-200) retains binding to the NPC, while the smaller, inactive fragments lose NPC binding (FIG. 15C).

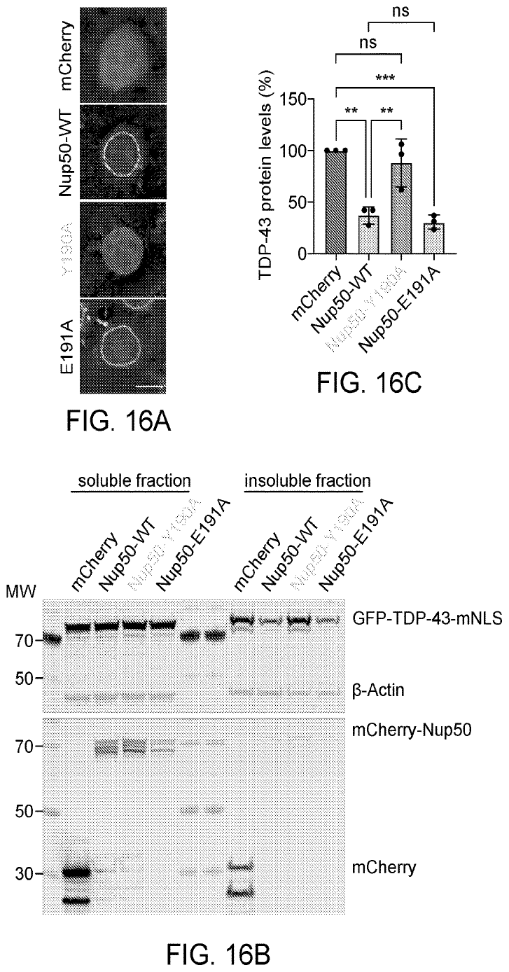

Single amino acid changes in full-length Xenopus Nup50 were shown to decrease NPC-binding (Holzer et al., The EMBO Journal 40:e108788 (2021)); two of the variants that decreased NPC binding (Y190A) and one that had no effect on NPC binding (E191A) were translated to full-length human Nup50. As in Xenopus Nup50, human Nup50 with the Y190A substitution has decreased NPC binding, while human Nup50 E191A maintains NPC binding (FIG. 16A). Disrupting NPC-association abrogated Nup50 polypeptide activity towards insoluble TDP-43 (FIGS. 16B and 16C).

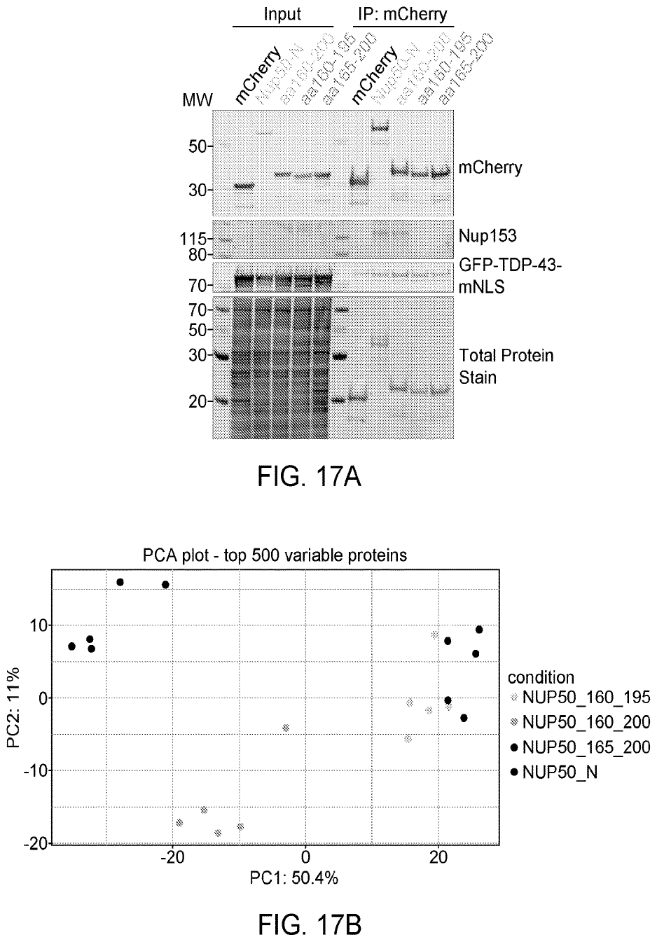

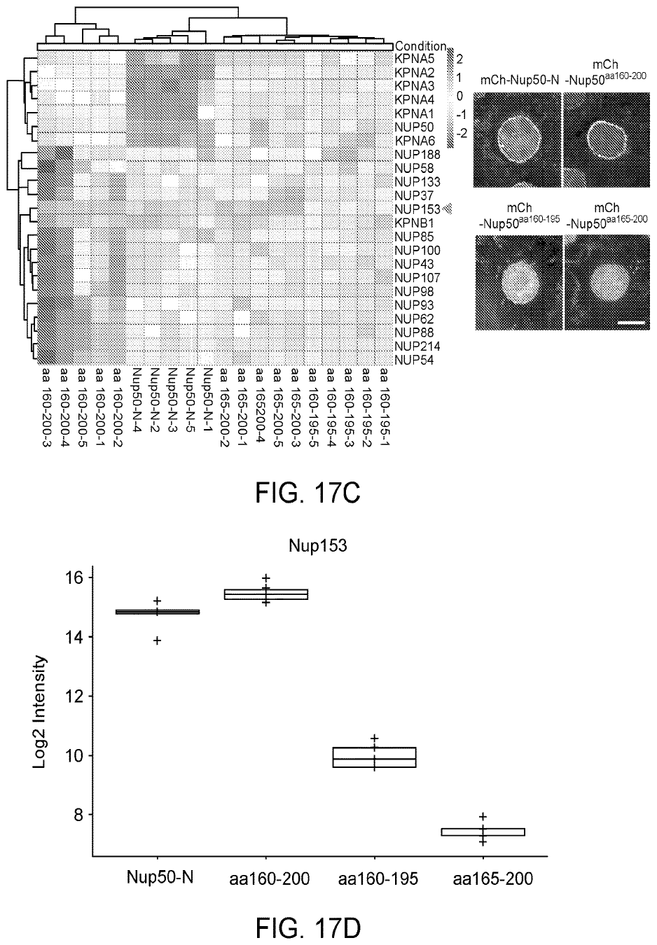

Interactions of active (Nup50-N and aa160-200) and inactive (aa160-195 and aa165-200) Nup50 polypeptide fragments were evaluated based on immunoprecipitation (IP) and mass-spec (MS) experiments. Active and inactive fragments interacted with TDP-43. Only active Nup50 fragments interacted with Nup153 (FIG. 17A). PCA plot showed that data quality was good (FIG. 17C). Active Nup50 fragments interacted more with importins and nucleoporins than inactive fragments (FIGS. 17C and 17D).

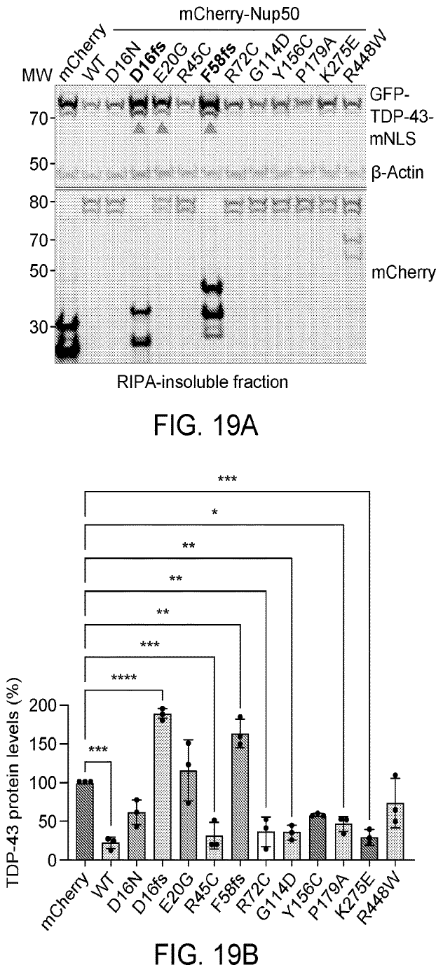

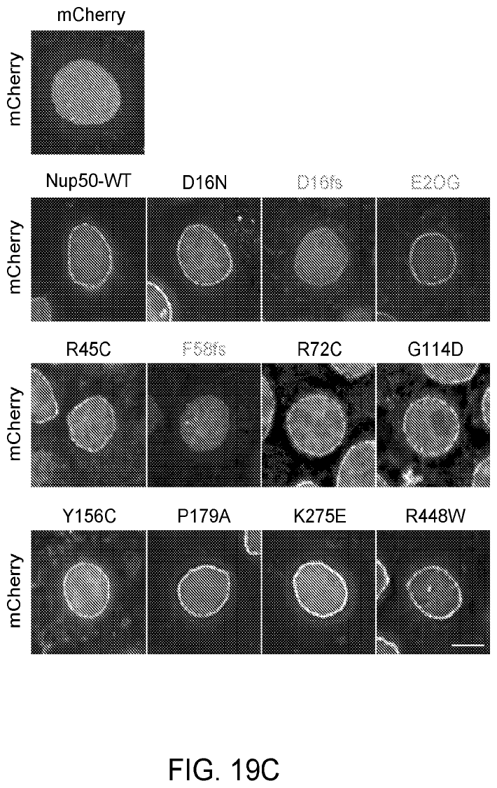

Several mutations in Nup50 were recently linked to ALS (Megat et al., Nat. Commun., 14:342 (2023); FIG. 18A). Patient-derived mutations in Nup50 polypeptides reduced activity towards TDP-CTF aggregation (FIGS. 18B-18C) and TDP-43-mNLS (FIGS. 19A and 19B). ALS-linked Nup50 frame-shift mutants did not display NPC binding (FIG. 19C).

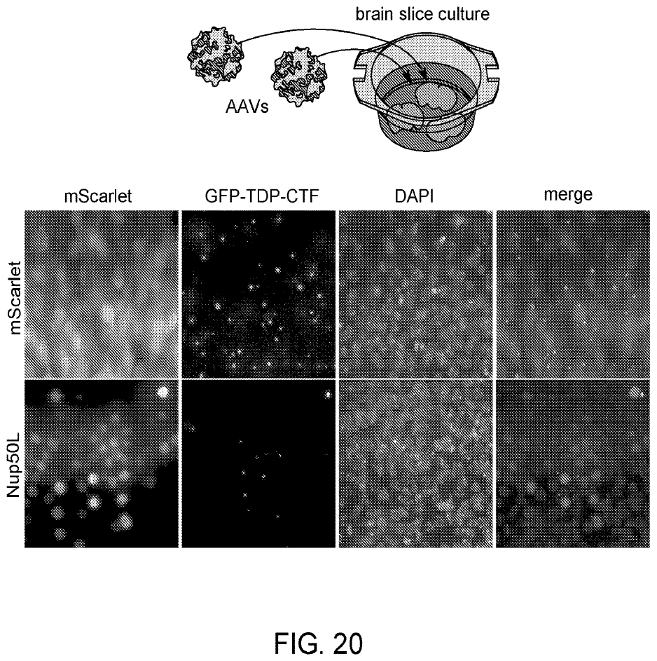

mCherry-tagged Nup50 polypeptide and a GFP-TDP-CTF polypeptide were co-expressed in ex vivo mouse brain slice cultures using an AAV-mediated expression system. Nup50 polypeptide expression reduced TDP-CTF aggregation in organotypic brain slice cultures (FIG. 20).

### Example 7: Treating ALS

A human identified as having, or as being at risk of developing, ALS is administered one or more Nup50 polypeptides or fragments thereof. The administered Nup50 polypeptides or fragments thereof can slow, delay, or prevent progression of neurodegeneration in the CNS (e.g., within the brain and/or spinal cord) of the human.

### Example 8: Treating ALS

A human identified as having, or as being at risk of developing, ALS is administered nucleic acid encoding a Nup50 polypeptide or a fragment thereof. The administered nucleic acid can encode the Nup50 polypeptide or the fragment thereof within the CNS (e.g., within the brain and/or spinal cord) of the human to slow, delay, or prevent progression of neurodegeneration in the CNS (e.g., within the brain and/or spinal cord) of the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a TAR DNA-binding protein 43 (TDP-43) proteinopathy, wherein said method comprises administering a nucleoporin 50 (Nup50) polypeptide or a fragment of said Nup50 polypeptide to said mammal.

2. The method of claim 1, wherein said Nup50 polypeptide or said fragment is effective to reduce a symptom of said TDP-43 proteinopathy.

3. The method of claim 2, wherein said symptom is selected from the group consisting of deterioration in behavior and personality, depression, apathy, social withdrawal, mood swings, irritability, aggressiveness, changes in sleeping habits, wandering, loss of inhibitions, delusions, emotional blunting, compulsive or ritualistic behavior, changes in eating habits or diet, deficits in executive function, lack of insight, agitation, emotional instability, difficulty producing or comprehending spoken or written language, memory loss, and symptoms of motor neuron disease such as muscle weakness, muscle atrophy, fasciculations, spasticity, dysarthria, and dysphagia.

4. The method of claim 1, wherein said method comprises identifying said mammal as being in need of said Nup50 polypeptide or said fragment prior to said administering step.

5. A method for reducing aggregation of TDP-43 polypeptides in a central nervous system (CNS) of a mammal having a TDP-43 proteinopathy, wherein said method comprises administering an Nup50 polypeptide or a fragment of said Nup50 polypeptide to said mammal.

6. The method of claim 1, wherein said method comprises administering said Nup50 polypeptide to said mammal.

7. The method of claim 1, wherein said method comprises administering said fragment of said Nup50 polypeptide to said mammal.

8. The method of claim 7, wherein said fragment of said Nup50 polypeptide consists of the amino acid sequence set forth in any one of SEQ ID NOs:3-11.

9. The method of claim 5, wherein said method comprises identifying said mammal as being in need of said Nup50 polypeptide or said fragment prior to said administering step.

10. A method for treating a mammal having a TDP-43 proteinopathy or reducing aggregation of TDP-43 polypeptides in a central nervous system (CNS) of a mammal, wherein said method comprises administering nucleic acid encoding a Nup50 polypeptide or a fragment of said Nup50 polypeptide to said mammal, wherein said Nup50 polypeptide or said fragment is expressed by cells in a central nervous system (CNS) of said mammal.

11. The method of claim 10, wherein said method comprises treating said mammal having said TDP-43 proteinopathy, and wherein said Nup50 polypeptide or said fragment is effective to reduce a symptom of said TDP-43 proteinopathy.

12. The method of claim 11, wherein said symptom is selected from the group consisting of deterioration in behavior and personality, depression, apathy, social withdrawal, mood swings, irritability, aggressiveness, changes in sleeping habits, wandering, loss of inhibitions, delusions, emotional blunting, compulsive or ritualistic behavior, changes in eating habits or diet, deficits in executive function, lack of insight, agitation, emotional instability, difficulty producing or comprehending spoken or written language, memory loss, and symptoms of motor neuron disease such as muscle weakness, muscle atrophy, fasciculations, spasticity, dysarthria, and dysphagia.

13. The method of claim 10, wherein said method comprises identifying said mammal as being in need of said nucleic acid prior to said administering step.

14-15. (canceled)

16. The method of claim 10, wherein said nucleic acid encodes said Nup50 polypeptide.

17. The method of claim 10, wherein said nucleic acid encodes said fragment of said Nup50 polypeptide.

18. The method of claim 17, wherein said fragment of said Nup50 polypeptide consists of the amino acid sequence set forth in any one of SEQ ID NOs:3-11.

19-21. (canceled)

22. The method of claim 1, wherein said mammal is a human.

23. The method of claim 1, wherein said TDP-43 proteinopathy is selected from the group consisting of frontotemporal dementia (FTD), amyotrophic lateral sclerosis (ALS), Alzheimer's disease, traumatic brain injury (TBI), chronic traumatic encephalopathy (CTE), limbic-predominant age-related TDP-43 encephalopathy (LATE), dementia with Lewy bodies (DLB), Parkinson's disease, Huntington's disease, argyrophilic grain disease (AGD), hippocampal sclerosis (HS), Guam ALS, Guam parkinsonism-dementia complex (G-PDC), Perry disease, facial onset sensory and motor neuronopathy (FOSMN), inclusion body myositis (IBM), oculopharyngeal muscular dystrophy (OPMD), and distal myopathies with rimmed vacuoles (DMRV).

24-30. (canceled)

31. The method of claim 7, wherein said fragment of said Nup50 polypeptide consists of the amino acid sequence set forth in any one of SEQ ID NOs:14-24.

32. The method of claim 17, wherein said fragment of said Nup50 polypeptide consists of the amino acid sequence set forth in any one of SEQ ID NOs:14-24.


SEQUENCE LISTING (24 sequences)

SEQ ID NO: 1 - DNA, 1323 bp, Homo sapiens
  source 1..1323: mol_type = other DNA
atggccagtgaggaagtcttgaagaatagagccataaagaaagcaaagcgcagaaatgttggatttgaatctgacactggaggagcctttaaaggttttaaaggtttggtggtaccttctggaggaggacgcttttctggatttggtagtggcgctggagggaagcctttggaaggactgtcgaatggaaacaacataaccagtgcccctcccttcgccagtgcaaaggcagcggcagatcccaaggtagcctttggttctcttgctgcaaatggccctaccaccttggttgataaagtttcaaatcccaaaactaatggggacagtcagcagccctcctcctctggccttgcttccagtaaagcttgtgtcggaaatgcctatcacaagcagttggccgccttgaactgctccgtgcgggattggatagtgaagcacgtgaatacaaaccccctctgtgatctgacacctatctttaaagactatgagaaatatttagcaaacattgaacagcaacacgggaacagtggcaggaattctgaaagtgaatctaacaaagtggcagctgaaacacagtctccttccctttttggctcaacaaaattacagcaagagtcaacgtttttgtttcatggcaacaaaactgaagatacacctgacaagaagatggaggtggcatctgaaaagaaaacggacccatcatcactaggagcgacaagtgcctcatttaatttcggcaagaaagttgatagctctgttttgggctcattaagctctgtccccctgactggattttctttctcccctggaaactccagtttatttggcaaagatactacccagagtaaaccagtctcttcaccatttcccactaaaccattggagggccaagcagaaggtgacagtggtgaatgcaaaggtggagatgaagaagagaatgatgagccacccaaagtagtagttaccgaagtaaaagaagaagatgctttttactccaaaaagtgtaaactgttttacaagaaagacaatgagtttaaagagaaaggcataggtactctgcatttaaaacctacagcaaatcagaagacacagcttttggtgcgggcagacaccaatttaggcaacatattgctgaacgttctgattccacccaatatgccatgtacgcgaacagggaagaataacgttcttatcgtctgtgttccaaatccaccaattgacgagaagaatgccaccatgccagtcaccatgttgattcgggtaaaaaccagcgaggatgcagacgagttgcacaaaattttactggagaaaaaggatgcctga

SEQ ID NO: 2 - DNA, 1407 bp, Homo sapiens
  source 1..1407: mol_type = other DNA
atggccaaaagaaatgccgagaaggaactgacagataggaattgggatcaagaagatgaagctgaagaggtgggaacattctccatggccagtgaggaagtcttgaagaatagagccataaagaaagcaaagcgcagaaatgttggatttgaatctgacactggaggagcctttaaaggttttaaaggtttggtggtaccttctggaggaggacgcttttctggatttggtagtggcgctggagggaagcctttggaaggactgtcgaatggaaacaacataaccagtgcccctcccttcgccagtgcaaaggcagcggcagatcccaaggtagcctttggttctcttgctgcaaatggccctaccaccttggttgataaagtttcaaatcccaaaactaatggggacagtcagcagccctcctcctctggccttgcttccagtaaagcttgtgtcggaaatgcctatcacaagcagttggccgccttgaactgctccgtgcgggattggatagtgaagcacgtgaatacaaaccccctctgtgatctgacacctatctttaaagactatgagaaatatttagcaaacattgaacagcaacacgggaacagtggcaggaattctgaaagtgaatctaacaaagtggcagctgaaacacagtctccttccctttttggctcaacaaaattacagcaagagtcaacgtttttgtttcatggcaacaaaactgaagatacacctgacaagaagatggaggtggcatctgaaaagaaaacggacccatcatcactaggagcgacaagtgcctcatttaatttcggcaagaaagttgatagctctgttttgggctcattaagctctgtccccctgactggattttctttctcccctggaaactccagtttatttggcaaagatactacccagagtaaaccagtctcttcaccatttcccactaaaccattggagggccaagcagaaggtgacagtggtgaatgcaaaggtggagatgaagaagagaatgatgagccacccaaagtagtagttaccgaagtaaaagaagaagatgctttttactccaaaaagtgtaaactgttttacaagaaagacaatgagtttaaagagaaaggcataggtactctgcatttaaaacctacagcaaatcagaagacacagcttttggtgcgggcagacaccaatttaggcaacatattgctgaacgttctgattccacccaatatgccatgtacgcgaacagggaagaataacgttcttatcgtctgtgttccaaatccaccaattgacgagaagaatgccaccatgccagtcaccatgttgattcgggtaaaaaccagcgaggatgcagacgagttgcacaaaattttactggagaaaaaggatgcctga

SEQ ID NO: 3 - protein, 468 aa, Homo sapiens
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 4 - protein, 440 aa, Homo sapiens
MASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 5 - protein, 440 aa, synthetic construct; identical to SEQ ID NO: 4

SEQ ID NO: 6 - protein, 210 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSES

SEQ ID NO: 7 - protein, 164 aa, synthetic construct
NVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSES

SEQ ID NO: 8 - protein, 61 aa, synthetic construct
ACVGNAYHKQLAALNCSVRDWIVKHVNTNPLCDLTPIFKDYEKYLANIEQQHGNSGRNSES

SEQ ID NO: 9 - protein, 51 aa, synthetic construct
LAALNCSVRDWIVKHVNTNPLCDLTPIFKDYEKYLANIEQQHGNSGRNSES

SEQ ID NO: 10 - protein, 51 aa, synthetic construct
ACVGNAYHKQLAALNCSVRDWIVKHVNTNPLCDLTPIFKDYEKYLANIEQQ

SEQ ID NO: 11 - protein, 41 aa, synthetic construct
LAALNCSVRDWIVKHVNTNPLCDLTPIFKDYEKYLANIEQQ

SEQ ID NO: 12 - protein, 38 aa, synthetic construct
MAKRNAEKELTDRNWIGIGIKKMKLKMWEHSPWPVRKS

SEQ ID NO: 13 - protein, 93 aa, synthetic construct
MAKRNAEKELTDRNWDQEDEAEDVGTFSMASEEVLKNRAIKKAKRRNVGFESDTGGALKVLKVWWYLLEEDAFLDLVVALEGSLWKDCRMETT

SEQ ID NO: 14 - protein, 468 aa, synthetic construct
MAKRNAEKELTDRNWNQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 15 - protein, 468 aa, synthetic construct
MAKRNAEKELTDRNWDQEDGAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 16 - protein, 468 aa, synthetic construct
MAKRNAEKELTDRNWDQEDEAEDVGTFSMASEEVLKNRAIKKAKCRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 17 - protein, 468 aa, synthetic construct
MAKRNAEKELTDRNWDQEDEAEDVGTFSMASEEVLKNRAIKKAKRRNVGFESDTGGAFKGFKGLVVPSGGGCFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 18 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFDSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 19 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNACHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 20 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNALCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 21 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGEKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 22 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIWVKTSEDADELHKILLEKKDA

SEQ ID NO: 23 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDAEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 24 - protein, 468 aa, synthetic construct
MAKRNAEKELTDRNWDQEDEAEDVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYAKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA